(19) United States
(12) Patent Application Publication
(10) Pub. No.: US 2020/0355704 A1
(43) Pub. Date: Nov. 12, 2020

(54) TEST KITS AND ASSAYS

(71) Applicant: Otago Innovation Limited, Dunedin (NZ)

(72) Inventors: Alison Kay Heather, Dunedin (NZ); Stephen John Sowerby, Dunedin (NZ)

(21) Appl. No.: 16/761,183

(22) PCT Filed: Nov. 2, 2018
(86) PCT No.: PCT/NZ2018/050154

§ 371 Date: May 1, 2020

Related U.S. Application Data
(60) Provisional application No. 62/581,260, filed on Nov. 3, 2017.
(60) Provisional application No. 62/614,680, filed on Jan. 8, 2018.

(30) Foreign Application Priority Data
Aug. 27, 2018 (NZ) 745713

(51) Int. Cl. G01N 33/74; C12Q 1/6897

(52) U.S. Cl. CPC G01N 33/743; G01N 2333/723; C12Q 1/6897

(57) ABSTRACT

The present invention provides in vitro test kits, assays and methods useful for screening a test sample for the presence of a ligand which is characterized by its ability to form a complex with a steroid hormone receptor and elicit a genomic response when in a cell. Advantageously, the activity-based assays which form the basis of the test kits and methods described herein, are particularly useful in detecting the presence of a ligand of unknown structure, for example, a designer drug used by equine, canine and human athletes in sports doping. Different assay prototypes are disclosed in which activation of the hormone receptor by a ligand binding interaction may be detected, for example, though activation of a reporter molecule. In certain examples, the present invention provides test kits, assays and methods involving aptame: fluorophore reporter constructs for detection of a ligand from (e.g.) a sample taken trackside from an athlete.

### TECHNICAL FIELD

The invention relates generally to assays, methods and test kits for detection of a ligand in a test sample. In particular, the present invention provides assays, methods and test kits to screen a test sample for the presence of a ligand which ligand is characterized by its ability to form a complex with a steroid hormone receptor and elicit a genomic response when in a cell.

### BACKGROUND OF THE INVENTION

The detection of ligands that bind to steroid hormone receptor proteins is important in many areas including, for example, environmental testing for organic pollutants that have the potential to affect human health, diagnostic and prognostic testing for human disease including (e.g.) detection and/or monitoring of endocrine- and non-endocrine cancers, as well as in drug testing including professional sports organisations charged with maintaining fair competition in (e.g.) human athletes and in the racehorse industry.

A common way to detect the presence of ligands that bind to steroid hormone receptor proteins is to measure them directly in a sample. However, samples are often complex mixtures of molecules and typically require a complicated process of preparation for analysis. Detecting the presence of ligands in a sample typically relies on processes such as liquid or gas chromatography to separate the molecular species from a complex mixture into fractions of relatively pure composition to then analyse each fraction with a structure-sensitive method such as mass spectrometry. Automated purification systems, gas or liquid chromatograms, and mass spectrometers are costly and technically complicated laboratory instruments that must be continually calibrated and operated by trained technicians in order to produce reliable results. Another disadvantage is that this methodology does not provide information about the biological activity of a ligand and is therefore unable to differentiate between biologically active and biologically inactive ligand molecules. Additionally, this structure-based methodology is unable to distinguish between agonists and antagonists. Furthermore, prior knowledge of the molecular structure of the ligand(s) and its associated metabolite(s) due to the biological metabolism of the ligand(s) is often required to achieve reliable identification of the presence of ligand(s) in the sample.

In addition to spectrometry based detection methods, some cell-based assays for the detection of presence of ligand/s that bind to steroid hormone receptor proteins have been developed. However, there are significant limitations associated with these cell-based assays that also require specialised equipment and expertise to maintain living cell cultures. This increases the cost of cell-based testing and reduces widespread application of these methods. Additionally, the high level of molecular complexity of a living cell makes testing difficult and reduces sensitivity and reproducibility.

In a sample for analysis it is often advantageous to detect the presence of ligand molecules that bind to steroid hormone receptors and modulate gene expression. For example, ligands that bind to androgen receptor and elicit a genomic response are associated with anabolic growth effects. Anabolic growth in animals and humans can provide advantages in terms of (e.g.) muscle mass, bone remodelling, elevated blood production, increased appetite and strength, conditioning, endurance and exercise recovery, but can also be an indication for disease. Detecting the presence of ligands that bind to androgen receptors and cause anabolic effects has particular industrial applications in monitoring biological samples for reasons including: detecting prohibited drug use involving animals such as race horses and dogs, as well as humans and human athletes; detecting prohibited additives in food or food supplements; for detecting the presence of prohibited ligands used to stimulate growth in animals used for food for example involving cattle, sheep, pigs, chickens and fish; screening for drugs; assessing health status.

Steroid hormone receptors contain binding domains that specifically bind to molecular ligands, which causes the steroid hormone receptor-ligand complex to initiate a variety of cellular responses including genomic responses, where the expression of genes is directly modulated by the steroid hormone receptor.

The present invention is specifically concerned with detecting the presence and/or potency of ligands which are characterized by their ability to directly modulate gene expression when in a cell through their binding to steroid hormone receptors.

### SUMMARY OF THE INVENTION

The inventions described and claimed herein have many attributes and examples including, but not limited to, those set forth or described or referenced in this Summary of the Invention. It is not intended to be all-inclusive and the inventions described and claimed herein are not limited to or by the features or examples identified in this Summary of the Invention, which is included for purposes of illustration only and not restriction.

In an aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid response element

wherein, the presence of a ligand in the test sample is determined when the sample is combined with the test kit and binding between the receptor-ligand complex and the nucleic acid response element is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to this aspect of the present invention, the test kit further comprises at least one steroid hormone receptor cofactor, steroid metabolism machinery, transcription and/or translation machinery and/or a cell-free extract as described herein.

In a related example, the steroid hormone receptor cofactor is selected from one or more of heat shock protein (HSP), including but not limited to, heat shock protein 70, heat shock protein 40, heat shock protein 90, p23, heat shock protein organizing protein (Hop), 48 kD Hip protein, p60, and FKBP52.

In a further related example according to this aspect of the present invention, the test kit further comprises HSP90.

In another example according to this aspect of the present invention, the nucleic acid response element is operably linked to a reporter construct, and binding of the receptor-ligand complex to the nucleic acid response element is detected by measuring transcription or translation of the reporter construct.

In another example, the reporter construct is comprised of a promoter sequence and a reporter gene.

In a further example, transcription from the promoter sequence is activated when the receptor-ligand complex binds to the nucleic acid response element.

In a further example, the reporter construct comprises a sequence encoding an RNA aptamer capable of binding to a fluorophore. In a related example, binding of the receptor-ligand complex to the nucleic acid response element is detected by transcription of the RNA aptamer, where upon binding of the fluorophore to the RNA aptamer fluorescence of the fluorophore is detected.

In yet a further example, the reporter construct is selected from the group consisting of a gene encoding any protein or polypeptide, a gene that does not encode a protein or polypeptide and a synthetic nucleic acid sequence that either does or does not encode a protein or polypeptide. In a related example, the reporter construct is selected from a gene or nucleic acid sequence encoding a fluorescent protein including, but not limited to, green fluorescent protein, red fluorescent protein and yellow fluorescent protein, a gene encoding β-galactosidase including but not limited to LacZ, β-glucuronidase (GUS), alkaline phosphatase, luciferase, amino acid biosynthetic genes, e.g., the yeast LEU2, HIS3, or LYS2 genes, nucleic acid biosynthetic genes, e.g., URA3 or ADE2 genes, the chloramphenicol acetyltransferase (CAT) gene, or any surface antigen gene for which specific antibodies are available.

In another example, the test kit further comprises translation and/or transcription machinery to facilitate transcription and/or translation of the reporter construct.

In a further example, binding of the receptor-ligand complex to the nucleic acid response element is detected using methods including, but not limited to, optical methods, spectroscopy, visible spectroscopy, Raman spectroscopy, UV spectroscopy, surface plasmon resonance, electrochemical methods, impedance, resistance, capacitance, mechanical sensing by changes in mass, changes in mechanical resonance, electrophoresis, gel electrophoresis, gel retardation, imaging, fluorescence, fluorescence resonance energy transfer, polymerase chain reaction (PCR) quantitative PCR (also known as real-time PCR, qPCR), reverse transcription PCR (RT-PCR) and reverse transcription qPCR (RTqPCR).

In another aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid response element

wherein the relative amount of steroid hormone receptor to nucleic acid response element in the test kit is x:1, where x is the amount of steroid hormone receptor and is defined as [0.2≤x≤20], and wherein the presence of a ligand in the test sample is determined when the sample is combined with the test kit and binding of the receptor-ligand complex to the nucleic acid response element is detected.

In an example according to this aspect of the present invention, the relative amount of steroid hormone receptor to nucleic acid response element in the test kit is x:1, where x is the amount of steroid hormone receptor and is defined as [0.3≤x≤7].

In yet another aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid response element; and
(iv) at least one steroid hormone receptor cofactor selected from heat shock protein 90, heat shock protein 70, heat shock protein 40, p23, heat shock protein organizing protein (Hop), 48 kD Hip protein, p60, and FKBP52

wherein the presence of a ligand in the test sample is determined when the sample is combined with the test kit and binding of the receptor-ligand complex to the nucleic acid response element is detected.

In yet another aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a detection means for detecting binding between the receptor-ligand complex to the nucleic acid response element; and
(iv) at least one steroid hormone receptor cofactor selected from heat shock protein 90, heat shock protein 70, heat shock protein 40, p23, heat shock protein organizing protein (Hop), 48 kD Hip protein, p60, and FKBP52

wherein the relative amount of steroid hormone receptor to nucleic acid response element in the test kit is x:1, where x is the amount of steroid hormone receptor and is defined as [0.2≤x≤20], and wherein the presence of a ligand in the test sample is determined when the sample is combined with the test kit and binding of the receptor-ligand complex to the nucleic acid response element is detected.

In an example according to this aspect of the present invention, the relative amount of steroid hormone receptor to nucleic acid response element in the test kit is x:1, where x is the amount of steroid hormone receptor and is defined as [0.3≤x≤7].

In another aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex;
(iii) a reporter construct operably linked to the nucleic acid response element

wherein, the reporter construct is activated when the receptor-ligand complex binds to the nucleic acid response element

and wherein, the presence of a ligand in the test sample is determined when the sample is combined with the test kit and transcription of the reporter construct is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to this aspect of the present invention, the test kit further comprises a steroid hormone receptor cofactor including, but not limited to, heat shock protein 90, heat shock protein 70, heat shock protein 40, p23, heat shock protein organizing protein (Hop), 48 kD Hip protein, p60, and FKBP52.

In yet another example according to this aspect of the present invention, the test kit further comprises, in combination or in isolation, steroid metabolism machinery, transcription and/or translation machinery and/or a cell-free extract as described herein.

In a further example according to this aspect of the present invention, the reporter construct comprises a fluorophore binding RNA aptamer.

In another example according to this aspect of the present invention, the RNA aptamer is selected from Spinach, iSpinach and Broccoli, and the fluorophore which binds to the RNA aptamer thereby generating a fluorescent signal is 3,5-difluoro-4-hydroxybenzylidene imidazolinone (DFHBI).

In another example according to this aspect of the present invention, the RNA aptamer is Mango, and the fluorophore which binds to the RNA aptamer thereby generating a fluorescent signal is a derivative of thiazole orange (TO).

In yet a further example according to this aspect of the present invention, the nucleic acid response element and the reporter construct are comprised on the same nucleic acid sequence. In a related example, the nucleic acid sequence comprising the nucleic acid response element and the reporter construct is defined by SEQ ID NO: 19.

In yet another example according to this aspect of the present invention, the test kit further comprises a detection means for detecting transcription of the reporter construct.

In a further aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a reporter construct operably linked to the nucleic acid response element; and
(iv) transcription machinery

wherein, the reporter construct is activated when the receptor-ligand complex binds to the nucleic acid response element

and wherein, the presence of a ligand in the test sample is determined when the sample is combined with the test kit and transcription of the reporter construct is detected.

In an example according to this aspect of the present invention, the relative amount of steroid hormone receptor to nucleic acid response element in the test kit is x:1, where x is the amount of steroid hormone receptor and is defined as [0.2≤≤20]. In a related example, the relative amount of steroid hormone receptor to nucleic acid response element in the test kit is x:1, where x is the amount of steroid hormone receptor and is defined as [0.3≤x≤7].

In an example according to these and other aspects of the present invention, the steroid hormone receptor cofactor is selected from one or more of heat shock protein (HSP), including but not limited to, heat shock protein 70, heat shock protein 40, heat shock protein 90, p23, heat shock protein organizing protein (Hop), 48 kD Hip protein, p60, and FKBP52.

In a further related example according to these and other aspects of the present invention, the test kit further comprises HSP90.

In another example according to these and other aspects of the present invention, transcription of the reporter construct comprises detecting the levels of deoxyribose nucleic acid (DNA), messenger ribose nucleic acid (mRNA) or complementary deoxyribose nucleic acid (cDNA).

In a further example according to these and other aspects of the present invention, the nucleic acid response element and the reporter construct are contained on the same nucleic acid molecule.

In a further aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a reporter construct operably linked to the nucleic acid response element,

wherein, the reporter construct is activated when the receptor-ligand complex binds to the nucleic acid response element

and wherein, the presence of a ligand in the test sample is determined when the sample is combined with the test kit and translation of the reporter construct is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to this aspect of the present invention, the test kit further comprises a steroid hormone receptor cofactor including, but not limited to, heat shock protein 90, heat shock protein 70, heat shock protein 40, p23, heat shock protein organizing protein (Hop), 48 kD Hip protein, p60, and FKBP52.

In yet another example according to this aspect of the present invention, the test kit further comprises, in combination or in isolation, steroid metabolism machinery, transcription and/or translation machinery and/or a cell-free extract as described herein.

In yet another example according to this aspect of the present invention, the test kit further comprises a detection means for detecting translation of the reporter construct.

In yet a further aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a reporter construct operably linked to the nucleic acid sequence; and
(iv) transcription and translation machinery

wherein, the reporter construct is activated when the receptor-ligand complex binds to the nucleic acid response element

and wherein, the presence of a ligand in the test sample is determined when the sample is combined with the test kit and translation of the reporter construct is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is the amount of steroid hormone receptor and is defined as [0.3≤x≤7].

In an example according to these and other aspects of the present invention, the steroid hormone receptor cofactor is selected from one or more of heat shock protein (HSP), including but not limited to, heat shock protein 70, heat shock protein 40, heat shock protein 90, p23, heat shock protein organizing protein (Hop), 48 kD Hip protein, p60, and FKBP52.

In a further related example according to these and other aspects of the present invention, the test kit further comprises HSP90.

In another example according to these and other aspects of the present invention, translation of the reporter element comprises detecting the presence of a translated reporter protein.

In a further example according to these and other aspects of the present invention, the nucleic acid sequence and the reporter element are contained on the same nucleic acid molecule.

In a further aspect of the present invention there is provided an assay method for screening a test sample for the presence of a ligand, which ligand is capable of forming a complex with a steroid hormone receptor and eliciting a genomic response when in a cell, the assay method comprising the steps of:

(i) providing assay reagents comprising:
(a) a steroid hormone receptor that forms a receptor-ligand complex with a ligand from the test sample; and
(b) a nucleic acid response element that is bound by the receptor-ligand complex; and
(c) a detection means for detecting binding between the receptor-ligand complex and the nucleic acid response element; and

(ii) combining the test sample with the assay reagents

wherein, the presence of a ligand in the test sample is determined when the sample is combined with the test kit and binding between the receptor-ligand complex and the nucleic acid response element is detected.

In an example according to the assay methods described herein, the method further comprises comparing the test result to a reference threshold, where the reference threshold reflects the level of signal caused by steroid hormone receptor binding to the nucleic acid response element in the absence of ligand. In a related example, the presence of a ligand in the test sample is confirmed where the level of signal obtained from the test sample is higher than the level of signal obtained from the reference threshold.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In an example according to this aspect of the present invention, the assay reagents further comprise a steroid hormone receptor cofactor, steroid metabolism machinery, transcription and/or translation machinery and/or a cell-free extract as described herein.

In a further example according to this aspect of the present invention, the steroid hormone receptor cofactor is selected from one or more of heat shock protein (HSP), including but not limited to, heat shock protein 70, heat shock protein 40, heat shock protein 90, p23, heat shock protein organizing protein (Hop), 48 kD Hip protein, p60, and FKBP52.

In a further related example according to this aspect of the present invention, the heat shock protein is HSP90.

In an example according to the test kits and methods described herein, the steroid hormone receptor is selected from the group consisting of an androgen receptor (AR); estrogen receptor (ER) including, but not limited to, estrogen receptor-α (ER-α) and estrogen receptor-β (ER-β); progesterone receptor including, but not limited to, progesterone receptor A (PRA) and progesterone receptor B (PRB); mineralocorticoid receptor (MR); and glucocorticoid receptor (GR).

In another example according to the test kits and methods described herein, the ligand is a performance enhancing designer drug and/or steroid.

In a related example according to the test kits and methods described herein, the ligand is of an unknown chemical structure.

In a further example according to the test kits and methods described herein, the ligand is of a previously unknown chemical structure.

In yet another example according to the test kits and methods described herein, the biological sample is derived from an animal selected from the group consisting of equine, canine, camelid, bovine, porcine, ovine, caprine, avian, simian, murine, leporine, cervine, piscine, salmonid, primate, simian and human.

In yet another example according to the test kits and methods described herein, the test sample is derived from biological material selected from the group consisting of urine, saliva, stool, hair, tissues including, but not limited to, blood (plasma and serum), muscle, tumors, semen, etc.

In yet another example according to the test kits and methods described herein, the test sample is derived from a food selected from the group consisting of vegetable, meat, beverage including but not limited to sports drink and milk, supplements including, but not limited to, food supplements and sports supplements, nutritional supplements, herbal extracts, etc.

In yet another example according to the test kits and methods described herein, the test sample is derived from a medication selected from the group consisting of drug, tonic, syrup, pill, lozenge, cream, spray and gel.

In yet another example according to the test kits and methods described herein, the sample is derived from the environment selected from the group consisting of liquid, water, soil, textile including, but not limited to, plastics and mineral.

In another aspect of the present invention there is provided a test kit for screening a test sample for the presence of a ligand, which ligand is capable of forming a complex with an androgen receptor and eliciting a genomic response when in a cell, the test kit comprising:
(i) an androgen receptor that forms a receptor-ligand complex with a ligand from a test sample; and
(ii) a nucleic acid response element that is bound by the receptor-ligand complex; and
(iii) a detection means for detecting binding between the receptor-ligand complex to the nucleic acid response element

wherein, the presence of a ligand in the test sample is determined when the sample is combined with the test kit and binding between the receptor-ligand complex and the nucleic acid response element is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to this aspect of the present invention, the test kit further comprises a steroid hormone receptor cofactor including, but not limited to, heat shock protein 90, heat shock protein 70, heat shock protein 40, p23, heat shock protein organizing protein (Hop), 48 kD Hip protein, p60, and FKBP52.

In a further related example according to this aspect of the present invention, the heat shock protein is HSP90.

In yet another example according to this aspect of the present invention, the test kit further comprises, in combination or in isolation, steroid metabolism machinery, transcription and/or translation machinery and/or a cell-free extract as described herein.

In an example according to this aspect of the present invention, the nucleic acid response element comprises the sequence set forth in SEQ ID NO: 1 or SEQ ID NO: 2.

In another example according to this aspect of the present invention, the ligand is a selective androgen receptor modulator (SARM) compound.

In another aspect of the present invention there is provided a test kit for screening a test sample for the presence of a ligand, which ligand is capable of forming a complex with an estrogen receptor and eliciting a genomic response when in a cell, the test kit comprising:
(i) an estrogen receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid sequence

wherein, the presence of a ligand in the test sample is determined when the sample is combined with the test kit and binding between the receptor-ligand complex and the nucleic acid response element is detected.

In an example according to this aspect of the present invention, the nucleic acid response element comprises the sequence set forth in any one of SEQ ID NOs: 3 to 6.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to this aspect of the present invention, the test kit further comprises a steroid hormone receptor cofactor including, but not limited to, heat shock protein 90, heat shock protein 70, heat shock protein 40, p23, heat shock protein organizing protein (Hop), 48 kD Hip protein, p60, and FKBP52.

In a further related example according to this aspect of the present invention, the heat shock protein is HSP90.

In yet another example according to this aspect of the present invention, the test kit further comprises, in combination or in isolation, steroid metabolism machinery, transcription and/or translation machinery and/or a cell-free extract as described herein.

In another example according to this aspect of the present invention, the ligand is a selective estrogen receptor modulator (SERM) compound.

In yet another aspect of the present invention there is provided a test kit for screening a test sample for the presence of a ligand, which ligand is capable of forming a complex with a progesterone receptor and eliciting a genomic response when in a cell, the test kit comprising:
(i) a progesterone receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid sequence

wherein, the presence of a ligand in the test sample is determined when the sample is combined with the test kit and binding between the receptor-ligand complex and the nucleic acid response element is detected.

In an example according to this aspect of the present invention, the nucleic acid response element comprises the sequence set forth in any one of SEQ ID NOs: 7 to 10.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to this aspect of the present invention, the test kit further comprises a steroid hormone receptor cofactor including, but not limited to, heat shock protein 90, heat shock protein 70, heat shock protein 40, p23, heat shock protein organizing protein (Hop), 48 kD Hip protein, p60, and FKBP52.

In a further related example according to this aspect of the present invention, the heat shock protein is HSP90.

In yet another example according to this aspect of the present invention, the test kit further comprises, in combination or in isolation, steroid metabolism machinery, transcription and/or translation machinery and/or a cell-free extract as described herein.

In a further aspect of the present invention there is provided a test kit for screening a test sample for the presence of a ligand, which ligand is capable of forming a complex with a mineralocorticoid receptor and eliciting a genomic response when in a cell, the test kit comprising:
(i) a mineralocorticoid receptor that forms a receptor-ligand complex with a ligand from the test sample; and
(ii) a nucleic acid sequence that is bound by the receptor-ligand complex; and
(iii) a detection means for detecting binding between the receptor-ligand complex and the nucleic acid sequence

wherein, the presence of a ligand in the sample is determined when the sample is combined with the test kit and binding between receptor-ligand complex and the nucleic acid sequence is detected.

In an example according to this aspect of the present invention, the nucleic acid response element comprises the sequence set forth in SEQ ID NO: 11 or SEQ ID NO: 12.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to this aspect of the present invention, the test kit further comprises a steroid hormone receptor cofactor including, but not limited to, heat shock protein 90, heat shock protein 70, heat shock protein 40, p23, heat shock protein organizing protein (Hop), 48 kD Hip protein, p60, and FKBP52.

In a further related example according to this aspect of the present invention, the heat shock protein is HSP90.

In yet another example according to this aspect of the present invention, the test kit further comprises, in combination or in isolation, steroid metabolism machinery, transcription and/or translation machinery and/or a cell-free extract as described herein.

In yet another aspect of the present invention there is provided a test kit for screening a test sample for the presence of a ligand, which ligand is capable of forming a complex with a glucocorticoid receptor and eliciting a genomic response when in a cell, the test kit comprising:
(i) a glucocorticoid receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid sequence

wherein, the presence of a ligand in the test sample is determined when the sample is combined with the test kit and binding between receptor-ligand complex and the nucleic acid sequence is detected.

In an example according to this aspect of the present invention, the nucleic acid response element comprises the sequence set forth in SEQ ID NO: 13 or SEQ ID NO: 14.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to this aspect of the present invention, the test kit further comprises a steroid hormone receptor cofactor including, but not limited to, heat shock protein 90, heat shock protein 70, heat shock protein 40, p23, heat shock protein organizing protein (Hop), 48 kD Hip protein, p60, and FKBP52.

In a further related example according to this aspect of the present invention, the heat shock protein is HSP90.

In yet another example according to this aspect of the present invention, the test kit further comprises, in combination or in isolation, steroid metabolism machinery, transcription and/or translation machinery and/or a cell-free extract as described herein.

In yet another aspect of the present invention there is provided an assay method for screening a test sample for the presence of a ligand, which ligand is capable of forming a complex with a steroid hormone receptor and eliciting a genomic response when in a cell, the assay method comprising the steps of:

(i) providing assay reagents comprising:
(a) a steroid hormone receptor that forms a receptor-ligand complex with a ligand from the test sample; and
(b) a nucleic acid response element that is bound by the receptor-ligand; and
(c) a detection means for detecting binding between the receptor-ligand complex and the nucleic acid response element; and

(ii) combining the biological sample with the assay reagents

wherein, the relative amount of steroid hormone receptor to nucleic acid response element in the test kit is x:1, where x is the amount of steroid hormone receptor and is defined as [0.2≤x≤20], and wherein, the presence of a ligand in the sample is determined when the sample is combined with the test kit and binding between the receptor-ligand complex and the nucleic acid response element is detected.

In an example according to this aspect of the present invention, the relative amount of steroid hormone receptor to nucleic acid response element in the test kit is x:1, where x is the amount of steroid hormone receptor and is defined as [0.3≤x≤7].

In an example according to this aspect of the present invention, the test kit further comprises a steroid hormone receptor cofactor, steroid metabolism machinery, transcription and/or translation machinery and/or a cell-free extract as described herein.

In a further example according to this aspect of the present invention, the steroid hormone receptor cofactor is selected from one or more of heat shock protein (HSP), including but not limited to, heat shock protein 70, heat shock protein 40, heat shock protein 90, p23, heat shock protein organizing protein (Hop), 48 kD Hip protein, p60, and FKBP52.

In a further related example according to this aspect of the present invention, the heat shock protein is HSP90.

In yet another aspect of the present invention there is provided an assay method for screening a test sample for the presence of a ligand, which ligand is capable of forming a complex with a steroid hormone receptor and eliciting a genomic response when in a cell, the assay method comprising the steps of:

(i) providing assay reagents comprising:
(a) a steroid hormone receptor that forms a receptor-ligand complex with a ligand from the test sample; and
(b) a nucleic acid response element that is bound by the receptor-ligand complex; and
(c) a heat shock protein, including but not limited to heat shock protein 90; and
(d) a detection means for detecting binding between the receptor-ligand complex and the nucleic acid response element; and

(ii) combining the biological sample with the assay reagents

wherein, the relative amount of steroid hormone receptor to nucleic acid response element in the test kit is x:1, where x is the amount of steroid hormone receptor and is defined as [0.2≤x≤20], and wherein, the presence of a ligand in the sample is determined when the sample is combined with the test kit and binding of the receptor-ligand complex to the nucleic acid response element is detected.

In an example according to this aspect of the present invention, the relative amount of steroid hormone receptor to nucleic acid response element in the test kit is x:1, where x is the amount of steroid hormone receptor and is defined as [0.3≤x≤7].

In an example according to this aspect of the present invention, the test kit further comprises a steroid hormone receptor cofactor, steroid metabolism machinery, transcription and/or translation machinery and/or a cell-free extract as described herein.

In a further example according to this aspect of the present invention, the steroid hormone receptor cofactor is selected from one or more of heat shock protein (HSP), including but not limited to, heat shock protein 70, heat shock protein 40, heat shock protein 90, p23, heat shock protein organizing protein (Hop), 48 kD Hip protein, p60, and FKBP52.

In a further related example according to this aspect of the present invention, the heat shock protein is HSP90.

In yet another aspect of the present invention there is provided an article of manufacture for screening a test sample for the presence of a ligand, which ligand is capable of activating a steroid hormone receptor and eliciting a genomic response in a cell, the article of manufacture comprising a test kit as described herein together with instructions for how to detect the presence of a ligand in the sample.

In yet a further aspect of the present invention there is provided an article of manufacture for determining doping in an athlete, the article of manufacture comprising a test kit as described herein together with instructions for detecting the presence of a ligand in a sample derived from the athlete, wherein the presence of the ligand in the sample is indicative of doping in the athlete.

### BRIEF DESCRIPTION OF THE FIGURES

FIG. 1 shows the ARE/enhancer sequence (SEQ ID NO: 15). The non-italicized residues represent the enhancer sequence and italicized residues represent the ARE sequence with palindromes highlighted in bold.

FIG. 2 shows Testosterone-activated androgen receptor (AR) induces the transcription and translation of GFP. Coupled in vitro transcription and translation reactions were assembled using an ARE/enhancer-minProm-GFP DNA template. Reactions were assembled in 0.5 mL Eppendorf tubes, and initiated by addition of 4 nM testosterone. After 2 hours incubation, GFP was measured as increased fluorescence. *P<0.05 (Students t-test), T-liganded AR versus no AR control.

FIG. 3 shows GFP levels are greater in testosterone-induced reactions versus controls. Coupled in vitro transcription and translation reactions were assembled using the ARE/enhancer-minProm-GFP DNA template. Reactions (50 μL) were assembled in 0.5 mL Eppendorf tubes, and initiated by addition of 4 nM testosterone. After a 5 h incubation, GFP was measured as increased fluorescence. ****P<0.0001 (one-way ANOVA with Sidaks multiple comparison test).

FIG. 4 shows a freeze-dried version of the reaction components, stored on filter paper discs, is able to generate testosterone-induced GFP. Coupled in vitro transcription and translation reactions were assembled using the ARE/enhancer-minProm-GFP DNA template and immediately aliquoted onto paper discs, snap frozen and freeze dried, and stored at −80° C. Reconstituted reactions were assembled in 0.5 mL Eppendorf tubes using 50 μL nuclease-free water, and initiated by addition of 4 nM testosterone. After a 6 h incubation, GFP was measured as increased fluorescence.

FIG. 5 shows Testosterone specificity of the AR-regulated in vitro transcription/translation assay. Coupled in vitro transcription and translation reactions were assembled using the ARE/enhancer-minProm-GFP DNA template. Reactions (50 μL) were assembled in 0.5 mL Eppendorf tubes, and activated by 1 μM testosterone, 1 μM estradiol or 1 μM progesterone. After a 5 h incubation, GFP was measured as fluorescence.

FIG. 6 shows initial assessment of the sensitivity of the AR-regulated in vitro transcription/translation assay. Coupled in vitro transcription and translation reactions were assembled using the ARE/enhancer-minProm-GFP DNA template. Reactions (50 μL) were assembled in 0.5 mL Eppendorf tubes, and initiated by addition of testosterone across the sub μM to sub nM range. After a 5 h incubation, GFP was measured as fluorescence.

FIG. 7 shows an RNA agarose gel with RNA molecules generated by an IVT reaction, with the MMTV-luciferase template (plasmid >3000 bp DNA band). The gel shows that at ˜850 bp an RNA band is evident (expected size based on gene transcript length). The different lanes (left to right) show lane 1 (far left) DNA size marker, Lane 2 (MMTV IVT stimulated with testosterone), Lane 3 (MMTV IVT treated with ethanol), Lane 4 (column wash #1), Lane 5 (column wash #2).

FIG. 8 shows an RNA agarose gel with RNA molecules generated by an IVT reaction, with the ARE/enhancer DNA template (˜850 bp DNA band). The gel shows that at ˜450 bp an RNA band is evident. The different lanes (left to right) show lane 1 G44, Lane 2 G32, Lane 3 4 nM (1×) testosterone, Lane 4 40 nM (10×) testosterone, Lane 5 ethanol.

FIG. 9 shows Quant-IT RNA standard curve. RNA standards were incubated with the Quant-IT dye and fluorescence measured. This standard curve was used to calculate the RNA molecule concentration in the IVT reactions.

FIG. 10 shows the cyanine-5-labelled UTP (cy-5-UTP) and associated wavelength excitation and emission spectra.

FIG. 11 shows the synthetic ARE/enhancer GFP DNA sequence optimized for UTP and CTP (SEQ ID NO: 18).

FIG. 12 shows IVT reactions produce fluorescently labelled RNA molecules. IVT reactions with ARE/enhancer-synthetic DNA template were activated with testosterone (4 nM). The reactions were control (no label, and controls for autofluorescence), CY-5 labelled-CTP and CY-5-labelled UTP, CY-5 labelled CTP, or CY-5 labelled UTP. * P<0.005 (one way ANOVA with Sidaks multiple comparison test) double-labelled reaction versus control.

FIG. 13 shows IVT reactions were established with cy-5-labelled CTP and cy-5-labelled UTP, and ARE/enhancer synthetic DNA template. Reactions were activated with 4 nM testosterone (T), 40 nM T (10× T) or 0.1% (v/v) ethanol (E). RNA molecules were column purified before direct fluorescence measured. *P<0.05 ethanol versus Testosterone. *P<0.05 one way ANOVA with Sidaks post multiple comparison test.

FIG. 14 shows IVT reactions were established with cy-5-labelled CTP and cy-5-labelled UTP and MMTV-luciferase DNA template. Reactions were activated with 4 nM testosterone (T), 40 nM T (10× T) or 0.1% (v/v) ethanol (E). RNA molecules were column purified before direct fluorescence measured. *P<0.05 ethanol versus testosterone, Students t-test between E and T. n=1 for 10× T.

FIG. 15 shows an assessment of varying concentrations of the IVT core components. IVT reactions were prepared with different concentrations of NTP (1 and 10 nM), MgCl₂ (3, 5 and 7.5 nM), AR (9.1, 18, 36, 92, and 182 nM, with DNA concentration held at 12.8 nM) and the MMTV-luciferase DNA template (12.8, 25.6, 51.2 and 102.4 nM, with AR concentration held at 18 nM). Reactions were activated with 4 nM testosterone. RNA molecules were column purified before direct quantitation. The ratio of AR to DNA (when AR was varied) for the IVT reaction was 0.7, 1.4, 2.8, 7.1 and 14.2. The ratio of AR to DNA (when DNA was varied) for the IVT reaction was 1.4, 0.7, 0.35 and 0.18. All reactions produced RNA output however an AR:DNA ratio of 2.8 or less were the most efficient, although once the ratio reaches 0.35 or below there is a dramatic decrease in response. A high AR to DNA ratio (≥7.1) and a low AR to DNA ratio (≤0.18) were shown to be adverse for the IVT reaction.

FIG. 16 shows IVT reactions with cy-5-NTP labelled RNA molecules as output are able to show significant differences between gelding plasma samples of known low (G32) and high (G44) bioactivity. *P<0.05 Students t-test comparing G32 with G44.

FIG. 17 shows detection of testosterone-induced RNA synthesis using RTqPCR. IVT reactions were prepared with MMTV-luciferase DNA template. The reactions were activated with testosterone (4 nM) or ethanol (E, 0.1% v/v) and incubated for 2 h at 30° C. The RNA molecules were purified, treated with DNase, before RTqPCR used to measured RNA levels. A lower cycle threshold indicates there was more RNA in testosterone-treated reactions. (***p<0.0005 Students t-test).

FIG. 18 shows comparative measurements between commercial HeLa nuclear extract and in-house preparations of HeLa-PC-3, HuH7 and HEK293 nuclear extracts. IVT reactions were prepared with MMTV-luciferase as the DNA template and HeLa cell extract from a commercial source (Promega), or from in-house nuclear extracts prepared from cultures of HeLa, PC3 (prostate), HuH7 (liver) or HEK293 (kidney) cells. Once prepared, the IVT reactions were activated with testosterone (4 nM) and incubated for 2 h. RNA was purified, DNA template destroyed by DNase, then RTqPCR used to measure RNA levels.

FIG. 19 shows Testosterone-induced activation of AR mediated IVT reactions in the presence of in-house nuclear extracts. All four nuclear extracts tested showed the ability to generate more RNA transcript when activated by testosterone (T) versus ethanol (E) as indicated by a decrease in cycle threshold number. A lower threshold number demonstrates a higher level of RNA transcript. HeLa (cervical cancer cells), HuH7 (liver cells), HEK293 (kidney cells), and PC3 (prostate cancer cells).

FIG. 20 shows the IVT reaction can be stimulated by testosterone and its active metabolite, dihydrotestosterone (DHT). Each IVT-RTqPCR was completed 3 times (n=3). Y-axis shows cycle threshold with a decrease in cycle threshold representing more RNA transcripts.

FIG. 21 shows the ARE/enhancer DNA template immobilized on beads supports testosterone (T)-activated AR-induced IVT transcription of an RNA transcript, as measured by RTqPCR. RTqPCR was performed using a specific primer/probe set. E-ethanol, T-testosterone, G32-equine plasma, G44-equine plasma. Y-axis shows cycle threshold with a decrease in cycle threshold representing more RNA transcripts.

FIG. 22 shows IVT reactions measured by RTqPCR using stem-loop reverse primer approach. Testosterone (4 nM) activated IVT reactions comprised of the ARE/enhancer GFP template. The mRNA transcript was purified from the reaction, DNaseI-treated, and levels measured in a two-step RT-qPCR reaction involving a stem-loop reverse transcription primer, and a reverse PCR primer and probe specific for the stem-loop sequence. Y-axis shows cycle threshold with a decrease in cycle threshold representing more RNA transcripts.

FIG. 23 shows Testosterone-activated AR-ARE/enhancer IVT assay generates RNA Mango aptamer molecules that can be detected by TO1-PB fluorescence. Values shown are corrected for background TO1-PB fluorescence.

FIG. 24 shows the four different RNA Mango DNA templates were tested in IVT reactions that were either not activated (ethanol control) or activated with 4 nM testosterone (T). n=4 for Mango II, III and IV and n=1 for I.

FIG. 25 shows Testosterone-activated reactions produce more Mango II aptamer than the control reactions. IVT reactions were assembled and activated with testosterone (4 nM) or ethanol (baseline control). Triplicate reactions were completed on three consecutive days. Results are shown as mean±SEM, and are plotted on a log 10 scale. *P<0.05 Students t-test ethanol versus testosterone.

FIG. 26 shows IVT-Mango II reactions can detect subnM testosterone concentrations. IVT reactions were assembled and activated with testosterone ranging from 2 to 0.4 (nM) or ethanol (baseline control, 0 nM). Results are shown as mean±SEM, and are plotted on a log 10 scale.

FIG. 27 shows IVT-Mango reactions can detect androgenic activity in equine plasma samples. IVT reactions were assembled and activated with equine serum (15% v/v) from gelding racehorses, G44 and G32. Results are shown as mean±SEM.

FIG. 28 shows IVT-Mango reactions can detect four different SARMs and two different AAS. IVT reactions were activated with one of 6 androgenic molecules, or T. **P<0.01 *P<0.05 One way ANOVA with Sidaks multiple comparison test.

FIG. 29 shows Testosterone activated IVT reactions produce more iSpinach aptamer than the ethanol controls. IVT reactions were assembled and activated with testosterone (4 nM) or ethanol (baseline control). Triplicate reactions were completed on three consecutive days. Results are shown as mean±SEM.

FIG. 30 shows IVT-iSpinach reactions can detect androgenic activity in equine plasma samples. IVT reactions were assembled and activated with equine plasma samples, G33 or G44.

FIG. 31 shows AR binds to the AREGFP DNA template. From far right, protein ladder indicates size. Lane 1 is recombinant AR protein probed with AR antibody and secondary HRP antibody. Lane 2 is the AREGFP DNA template alone, with no AR added to the reaction mix. Lanes 3-5 show AR binds to AREGFP template in a MgCl₂ dependent manner. The next lanes 2-5 (SN after incubation) show the supernatant that was removed after the incubation reaction showing only very faint AR bands, indicating that most of the added AR was bound to the beads. The right hand lanes 2-5 show the beads after they have been heat treated (DNA would have separated from the beads) and washed. There are very faint AR bands indicating that most of the AREGFP DNA template/AR complex has been removed from the beads. Together, these negative controls show the specificity of the positive reactions and indicate that AR is binding to the AREGFP template immobilised onto beads.

FIG. 32 shows a dot blot of AR/ARE DNA template.

FIG. 33 shows green fluorescent protein (GFP) expression by the androgen receptor Assay Prototype 1 for a test reaction mix containing testosterone, as well as various positive and negative controls.

FIG. 34 shows androgen receptor Assay Prototype 1 versus androgen receptor Assay Prototype 0 testosterone dose response curves. Testosterone was serially diluted from 1×10⁻⁶M to 1×10⁻¹²M, and each concentration was measured for reporter protein output using either Prototype 0 or Prototype 1. A sigmoidal dose response curve was generated.

FIG. 35 shows the relative green fluorescent protein expression by the androgen receptor Assay Prototype 1 spiked with testosterone, estradiol and progesterone. These data demonstrate the specificity of the assay system for testosterone (i.e. androgen receptor specific ligand) versus estradiol and progesterone (non-androgen receptor specific ligands but known to cross-react and activate androgen receptors).

FIG. 36 shows a photograph of an illuminated agarose gel and the products of an IVT reaction using MMTV-luciferase DNA template, AR, HeLa cell extract, reaction buffer. The reaction was set up and AR activated with testosterone (100 ng). Controls included ethanol and testosterone in the absence of AR.

FIG. 37 shows an IVT reaction using MMTV-luciferase DNA template, AR, HeLa cell extract, reaction buffer. The reaction was set up and AR activated with testosterone at decreasing concentrations (100 ng, 50 ng, 25 ng, 12.5 ng, ethanol).

FIG. 38 shows IVT reaction using MMTV-luciferase DNA template, AR, in house HeLa cell extract, reaction buffer. The reaction was set up and AR activated with testosterone (100 ng). The control included ethanol. 10, 5 and 2.5 μl of RT product was tested in the PCR reaction

FIG. 39 shows a yeast cell androgen receptor Prototype Assay 0 that measures assay performance (β-galactosidase activity) as a function of cell density (OD₆₀₀=0.4 or OD₆₀₀=0.2) at both 30 and 60 minutes post activation using serum derived from a racehorse which had been administered with a pharmacological relevant dose of testosterone.

FIG. 40 shows an androgen receptor Prototype Assay 2 that measures RTqPCR cycle threshold as a function of HeLa cell extract concentration at 100, 75 and 50 μg/mL cell extract. Ethanol was included as a negative control.

FIG. 41 shows IVT-RT-PCR with testosterone (100 ng) versus ethanol.

FIG. 42 shows PCR of DNaseI treated DNA template.

FIG. 43 shows RT-PCR of IVT reactions. IVT reactions were performed (T, Ethanol, no NTPs). Turbo DNase was used to eliminate the DNA template before proceeding to RT-PCR. Results show DNA band for the positive IVT reaction, with T>ethanol (confirmed by RTqPCR). No NTPs (therefore no RNA can be formed) showed no band indicating the DNA template was destroyed. 4^{th} lane is PCR water control.

FIG. 44 shows an EC₅₀ plot of Testosterone (T) versus Dihydroteststerone (DHT) as measured by binding to, and activation of, androgen receptor. The Relative Potency of Dihydroteststerone to Testosterone was calculated at 2.3.

FIG. 45 shows Testosterone activates transcription of the iSpinach RNA aptamer molecule (A) 4×iSpinach DNA template (B) F30scaffoldiSpinach template. An in vitro transcription reaction was performed, followed by the addition of DFHBI and fluorescence buffer. Fluorescence was significantly increased in testosterone-activated reactions relative to either ethanol (vehicle, non-activated baseline) or no RNA generation controls. n=4 reactions (A), n=3 reactions (B).

FIG. 46 shows the effect of adding HSP90 to the reaction mixture. When HSP90 is not added to the reaction mixture (0 ng), then there is less ligand (testosterone) activation of AR, as shown by the smaller level of induction by testosterone over the ethanol control. However, when 100 ng HSP90 is included in the reaction mixture, then there is a marked increase in the activation of AR, as demonstrated by the increased level of induction by testosterone over the ethanol control. Finally, when 200 ng HSP90 is included in the reaction mixture, then there is almost complete suppression of non-liganded AR activation of RNA aptamer synthesis, and this blockade of AR by HSP90 is overcome when AR is activated by the ligand, testosterone.

### GENERAL DEFINITIONS

Unless specifically defined otherwise, all technical and scientific terms used herein shall be taken to have the same meaning as commonly understood by one of ordinary skill in the art (for example, in immunology, immunohistochemistry, protein chemistry, and biochemistry).

It is intended that reference to a range of numbers disclosed herein (e.g. 1 to 10) also incorporates reference to all related numbers within that range (e.g. 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

The term “and/or”, e.g., “X and/or Y” shall be understood to mean either “X and Y” or “X or Y” and shall be taken to provide explicit support for both meanings or for either meaning.

The term “a” or “an” refers to one or more than one of the entity specified; for example, “a receptor” or “a nucleic acid molecule” may refer to one or more receptor or nucleic acid molecule, or at least one receptor or nucleic acid molecule. As such, the terms “a” or “an”, “one or more” and “at least one” can be used interchangeably herein. Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### Selected Definitions

For the purposes of the present invention, the following terms shall have the following meanings.

The term “Assay Prototype 0” as used herein refers to yeast and mammalian cell-based assays for detection of a ligand that activates a steroid hormone receptor.

The term “Assay Prototype 1” as used herein refers to a cell-free assay for detection from a test sample of a ligand having steroid activity, which assay functions by determining reporter expression levels (e.g. green fluorescent protein) of a reporter element operably linked to a nucleic acid response element that is bound by a ligand activated receptor (or receptor-ligand complex), to detect the presence of a ligand.

The term “Assay Prototype 2” as used herein refers to a cell-free assay for detection from a test sample of a ligand having steroid activity, which assay functions by determining reporter transcript levels (e.g. mRNA or cDNA) of a reporter element operably linked to a nucleic acid that is bound by a ligand activated receptor (or receptor-ligand complex), to detect the presence of a ligand.

The term “Assay Prototype 3” as used herein refers to a cell-free assay for detection from a test sample of a ligand having steroid activity, which assays functions by determining a binding interaction between a ligand activated receptor (or receptor-ligand complex) and a nucleic acid sequence comprising an activated receptor binding domain, to detect the presence of a ligand.

The term “steroid hormone receptor” as used herein refers to a protein or polypeptide, including recombinant polypeptides that selectively binds to a ligand, which ligand is capable of activating the steroid hormone receptor, and includes, without limitation, an androgen receptor, an estrogen receptor, a progesterone receptor, a mineralocorticoid receptor and a glucocorticoid receptor. Typically, a steroid hormone receptor comprises a ligand binding domain, an activation domain and a deoxyribose nucleic acid binding domain. According to this definition, “steroid hormone receptor” may optionally include other cofactors, including (e.g.) heat shock proteins and the like, which help to hold the steroid hormone receptor in a folded and hormone responsive state for activation by a ligand.

The term “steroid hormone receptor cofactor” as used herein refers to one or more cofactors that help hold the steroid hormone receptor in an optimally folded and hormone responsive state for activation by a ligand.

The term “ligand” refers generally to any molecule that binds to a receptor, and includes without limitation, a polypeptide, a protein, a vitamin, a carbohydrate, a glycoprotein, a therapeutic agent, a drug, a glycosaminoglycan, or any combination thereof. As used herein, “ligand” includes, without limitation, steroid hormones, such as sex hormones including but not limited to estrogens, progestagens, androgens etc, as well as natural and synthetic derivatives and analogs and metabolites thereof, designer steroid hormones, androgenic anabolic steroids, and selective androgen-, progestagen- and estrogen receptor modulators, those that are currently known and those anticipated to be developed.

The term “receptor-ligand complex” and “activated steroid hormone receptor” as used herein refers to ligand bound to the steroid hormone receptor, where the steroid hormone receptor undergoes a structural transformation upon binding the ligand and is then said to be in an activated form. A receptor-ligand complex as described herein includes, without limitation, a monomer of a ligand bound hormone receptor (i.e. HR-L; where “HR” is the hormone receptor and “L” is the ligand), a dimer of a ligand bound hormone receptor (i.e. (HR-L)₂), a trimer of a ligand bound hormone receptor (i.e. (HR-L)₃) or some other higher order structure that would be apparent to a person skilled in the art.

The term “genomic response” as used herein refers to the ability of an activated steroid hormone receptor (i.e. a ligand bound receptor) to selectively bind to a nucleic acid binding motif and regulate the expression of a nucleic acid molecule, including genes, that are directly or indirectly linked to the binding motif. For the avoidance of doubt, the term “genomic response” need not refer to the regulation of genes within the nucleus of a cell, but rather that the response is one in which an activated hormone receptor has the ability to switch transcription of a nucleic acid sequence on or off, or to reduce or enhance the expression of a nucleic acid sequences, such as those contained within the reporter construct used in the test kits, assays and methods described herein.

The term “steroid metabolism machinery” as used herein refers to any enzyme or non-enzyme cofactor, and includes combinations of enzyme and non-enzyme cofactors, sufficient to convert a ligand from a physiologically inactive form to a physiologically active form or from a physiologically active form to a more physiologically active form, or from a physiologically active form to a less physiologically active form, or from a physiologically active form to a physiologically inactive form.

The term “cell-free extract” as used herein refers to an extract that is derived from a cell or a nucleus found within a cell, and is substantially free of any cell membrane component.

The term “detection means” as used herein refers to any apparatus, equipment or configuration adapted to detect the binding interaction between an activated steroid hormone receptor and nucleic acid response element. Examples of detection means include, but are not limited to, optical methods, spectroscopy, visible spectroscopy, Raman spectroscopy, UV spectroscopy, surface plasmon resonance, electrochemical methods, impedance, resistance, capacitance, mechanical sensing by changes in mass, changes in mechanical resonance, electrophoresis, gel electrophoresis, gel retardation, imaging, fluorescence and fluorescence resonance energy transfer, polymerase chain reaction etc.

The term “nucleic acid sequence” as used herein refers to a deoxyribose nucleic acid (DNA) sequence, a ribose nucleic acid sequence (RNA), messenger ribose nucleic acid (mRNA) and complementary DNA (cDNA), and is comprised of a continuous sequence of two or more nucleotides, also referred to as a polynucleotide.

The term “reporter construct” as used herein refers to a nucleic acid sequence encoding a reporter molecule that encodes an RNA or an enzyme or protein whose expression may be assayed; such RNA includes, but are not limited to, fluorophore binding aptamers, or synthetic RNA or mRNA, such proteins include, but are not limited to, green fluorescent protein (GFP), red fluorescent protein (RFP), orange fluorescent protein (OFP), β-galactosidase (LacZ), β-glucuronidase (GUS), alkaline phosphatase, luciferase, amino acid biosynthetic genes, e.g., the yeast LEU2, HIS3, or LYS2 genes, nucleic acid biosynthetic genes, e.g., URA3 or ADE2 genes, the chloramphenicol acetyltransferase (CAT) gene, or any surface antigen gene for which specific antibodies are available. Additionally, reporter genes may encompass any gene of interest whose expression product may be detected.

The term “promoter” as used herein is a nucleic acid sequence located proximal to the start of transcription at the 5′ end of an operably linked transcribed sequence. The promoter may contain one or more regulatory elements which interact in modulating transcription of the operably linked gene. The promoter is minimal in nature, including but not limted to, minimal CMV, minimal pA promoters, minimal pTKHSV promoters, minimal TATA-like promoters.

The term “operably linked” as used herein describes two macromolecular elements arranged such that modulating the activity of the first element induces an effect on the second element. In this manner, modulation of the activity of a promoter element may be used to alter and/or regulate the expression of an operably-linked reporter construct. For example, the transcription of a reporter construct that is operably-linked to a promoter element is induced by factors that “activate” the promoter's activity; transcription of a reporter construct that is operably-linked to a promoter element is inhibited by factors that “repress” the promoter's activity. Thus, a promoter region is operably-linked to the reporter construct if transcription of such a reporter construct is influenced by the activity of the promoter.

The term “expression” as used herein refers to the process by which the information encoded within a gene is expressed. If the gene encodes a protein, expression involves both transcription of the DNA into mRNA, the processing of the mRNA (if necessary) into a mature mRNA product, and translation of the mature mRNA into protein. A nucleic acid molecule, such as a deoxyribose nucleic acid (DNA) or gene is said to be “capable of expressing” a polypeptide (or protein) if the molecule contains the coding sequences for the polypeptide and the expression control sequences which, in the appropriate host environment, provide the ability to transcribe, process and translate the genetic information contained in the DNA into a protein product, and if such expression control sequences are operably-linked to the nucleotide sequence that encodes the polypeptide.

The term “sample” as used herein refers to any sample for which it is desired to test for the presence of a ligand.

The term “relative potency” or “RP” as used herein refers to the multiplier of biological activity exhibited by a test compound relative to a reference compound, where the biological activity is defined by the ability of the compound to bind to and activate a steroid hormone receptor (e.g.) as measured using the assays and test kits described herein. Where Relative Potency is >1, the test compound is more potent in terms of its biological activity as compared to the reference compound; where Relative Potency is <1, the test compound is less potent in terms of its biological activity as compared to the reference compound; and where Relative Potency=1, the test and reference compounds are equally potent in terms of their biological activities.

The term “activation factor” or “AF” as used herein relates to the measure of metabolic conversion of a test compound (e.g.) from a physiologically inactive state to a physiologically active state or from a less physiologically active state to a more physiologically active state. An activation factor >1 means that the test compound has undergone metabolic conversion to a more physiologically active state in the presence of metabolic machinery in the assay.

The term “reference threshold” as used herein means the level of assay activity measured in the absence of a test sample. In certain examples according to the inventions described herein, the reference threshold is determined using ethanol in place of test sample.

### DETAILED DESCRIPTION

The present invention provides test kits, assays and methods useful for screening a sample for the presence of a ligand, which ligand is capable of forming a complex with a steroid hormone receptor and eliciting a genomic response when in a cell. In certain examples, the inventions described herein have utility in the detection of performance enhancing pro/drugs (e.g. anabolic steroids) used in human as well as non-human athletes including race horses and dogs. In other examples, the inventions described herein have utility in screening foods and health food supplements for additives that may bind to a steroid hormone receptor and elicit a genomic response in a cell or do so following metabolic processing (i.e. in the case of so-called ‘prodrugs’).

The assays according to the present invention, on which the test kits and methods described herein are based, are fundamentally activity based assays which work on the principle of steroid hormone receptor activation through binding of a ligand derived from a sample to be tested. Activation of a steroid hormone receptor occurs when a ligand binds to the receptor and induces a conformational change in the tertiary structure of the protein, meaning that the receptor-ligand complex (also referred to herein as an ‘activated hormone receptor’) is then able to bind to a nucleic acid response element and elicit a so-called ‘genomic response’ (in other words the ability to up-/down-regulate genes in the nucleus of the cell which may then lead to an anabolic physiological effect). It is this binding interaction between the activated steroid hormone receptor and nucleic acid response element that is measured by the test kits, assays and methods described herein, as a proxy to detect the presence of a ligand having steroid-, or steroid-like activity in a sample being tested.

Accordingly, the test kits and assays according to the present invention have the ability to detect ligands of unknown structure such as (e.g.) ‘designer drugs’. Historically, this has not been possible since conventional laboratory testing equipment, such as gas/liquid chromatography and mass spectrometry, requires prior knowledge of the structure of the molecule being investigated.

As such, the activity assays according to the present invention overcome limitations associated with established prior art techniques.

Further, while the assays described herein are modeled on a molecular framework that largely mimics cell-based systems, the absence of cellular complexity provides in vitro systems with enhanced funtionality and improved assay sensitivity as compared to cell-based assays.

The various test kits and assays described herein each provide (i) a steroid hormone receptor inclusive of a ligand binding domain for binding a ligand that may be present in a sample to be tested and (ii) a nucleic acid response element comprising a protein binding domain which is bound by an activated steroid hormone receptor (or ligand-receptor complex; HR-L). The term ‘activated steroid hormone receptor’ refers to a receptor-ligand complex, and may include various permutations of the HR-L structure (e.g. monomer, dimer, trimer etc). The nucleic acid response element contains binding motif specific for the receptor-ligand complex. Accordingly, by combining the test kits and assays of the present invention with a test sample of interest, detection of a ligand, which possesses the potential to bind to a steroid hormone receptor and elicit a genomic response in a cell, is possible.

The terms “receptor binding domain”, “activated receptor binding domain”, “hormone receptor binding domain”, “activated hormone receptor binding domain”, “receptor-ligand binding domain” and “hormone receptor-ligand binding domain” are used interchangeably to refer to the protein binding domain of the nucleic acid response element that is bound by an activated hormone receptor or ligand-receptor complex, as defined herein.

The test kits, assays and methods described herein may further comprise (iii) a steroid hormone receptor cofactor as well as transcription and/or translation machinery, which additional cofactors and/or machinery enhances the overall performance of the assay. The steroid hormone receptor cofactor(s) as described herein helps hold the hormone receptor in an optimally folded and hormone responsive state for activation by a ligand, and largely prevents the steroid hormone receptor from binding to its response element. Examples of steroid hormone receptor cofactors according to the present invention, include but are not limited to, heat shock proteins including heat shock protein 70, heat shock protein 40, heat shock protein 90 and heat shock protein organizing protein (Hop), p23, 48 kD Hip protein, p60, and FKBP52.

The test kits, assays and methods described herein may further comprise (iv) steroid metabolism machinery sufficient to convert a ligand from a physiologically inactive form to a physiologically active form, or from a physiologically active form to a more physiologically active form or from a physiologically active form to a less physiologically active form, or from a physiologically active form to a physiologically inactive form. Only when the ligand is in a physiologically active form does it possess the ability to activate a steroid hormone receptor and elicit a genomic response. Accordingly, inclusion of steroid metabolism machinery in the test kits, assays and methods according to the present invention helps facilitate detection of physiologically inactive ligands from a test sample of interest, (e.g.) which ligands exist as pro-drugs (e.g. pro-hormones) and might otherwise evade detection using established methodologies. Furthermore, inclusion of steroid metabolism machinery in the test kits, assays and methods according to the present invention helps determine biological activity/potency of ligands necessary to show effect.

According to the present invention, the steroid hormone receptor cofactors, transcription and/or translation machinery as well as the steroid metabolism machinery may be provided in the test kits, assays and methods via a cell-free extract. In an example according to the present invention, the cell-free extract contains additional protein and non-protein enzymes, cofactors and the like, including (e.g.) additional steroid hormone receptor cofactors, transcription and/or translation machinery as well as the steroid metabolism machinery. In a further example, the cell-free extract is derived from a cell of interest. In a related example, the cell-free extract is derived from a cell in which the target ligand would be physiologically active. To better illustrate this point, and for the purpose of exemplification only, if the test kit according to the present invention has been configured to detect a performance enhancing androgen from a test sample obtained from a race horse, then the cell-free extract for inclusion in the test kit may be derived from the cell of an equine animal. That way the test kit is optimized for detection of the ligand of interest. However, this example does not preclude the possibility that the cell-free extract is derived from the cell of another species for which the target ligand is not necessarily physiologically active (e.g. in the example above, where the cell-extract is derived from a human cell line as opposed to a equine cell line).

Similarly, the steroid hormone receptor provided in the test kits, assays and methods according to the present invention is derived directly from the target species in which detection of a ligand of interest is to be interrogated. The term “derived from” used in this context includes a steroid hormone receptor that has been purified from the cell or nucleus of a cell of a target species of interest, or has been produced by synthetic or recombinant means (and may include one or more mutations to enhance (e.g.) ligand binding etc. Continuing the example referred to above, if the test kits, assays and methods according to the present invention are to be configured for possible detection of an androgen from the serum of a race horse, then the test kit will include an equine androgen receptor that has either (i) been purified from an equine cell or (ii) which has been produced by recombinant or synthetic means and would otherwise mimic the performance of an androgen receptor that has been purified from an equine cell.

Further, the present invention contemplates test kits in which some of the additional cofactor(s) and/or machinery is provided by a cell-free extract while other cofactor(s) and/or machinery are provided in a recombinant or synthetic form. By way of example only, the present invention further contemplates test kits comprising a cell-free extract which provides transcription and/or translation machinery as well as the steroid metabolism machinery, while the steroid hormone receptor cofactor (e.g. HSP90) is provided in a recombinant form.

According to the present invention, the cell-free extract is by definition subtantially free of any cell membrane material.

The test kits, assays and methods described herein may further comprise (v) a detection means for detecting binding between the receptor-ligand complex and the nucleic acid response element. This binding interaction may be measured indirectly via a reporter construct operably linked to the nucleic acid sequence (e.g. Assay Prototype 1 and Assay Prototype 2) or directly (e.g. Assay Prototype 3).

According to the test kits, assays and methods of the present invention, Assay Prototype 2 refers to a cell-free assay for detection from a test sample of a ligand which is capable of forming a complex with a steroid hormone receptor and eliciting a genomic response when in a cell, which assay functions by determining RNA transcript levels (e.g. mRNA or cDNA or synthetic RNA) of a reporter element operably linked to a nucleic acid that is bound by a ligand activated receptor (or receptor-ligand complex). Assay Prototype 1 refers to a cell-free assay for detection from a test sample of a ligand which is capable of forming a complex with a steroid hormone receptor and eliciting a genomic response when in a cell, which assay functions by determining gene expression levels (e.g. green fluorescent protein) of a reporter element operably linked to a nucleic acid that is bound by a ligand activated receptor (or receptor-ligand complex), to detect the presence of a ligand; and Assay Prototype 3 refers to a cell-free assay for detection from a test sample of a ligand which is capable of forming a complex with a steroid hormone receptor and eliciting a genomic response when in a cell, which assay functions by determining a binding interaction between a ligand activated receptor (or receptor-ligand complex) and a nucleic acid sequence comprising an activated receptor binding domain, to detect the presence of a ligand.

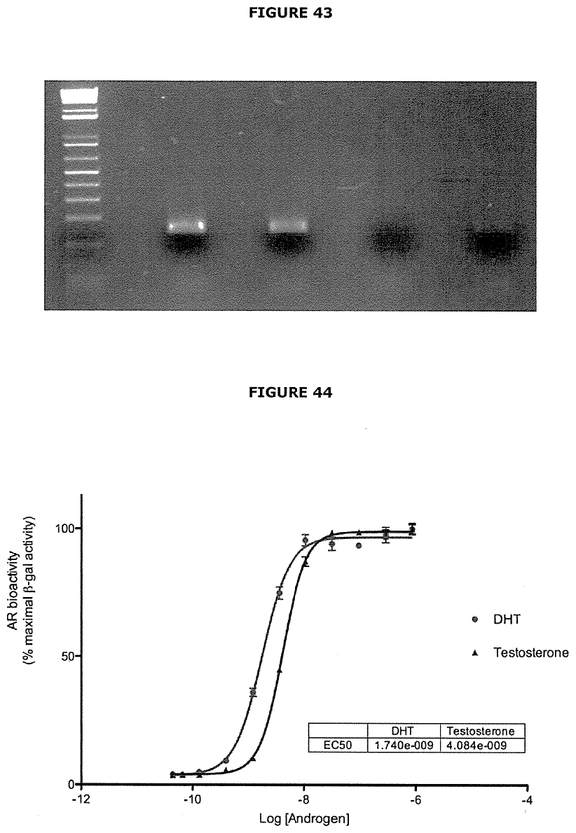

The test kits and assays according to the present invention are cell-free. This is particularly important since the molecular complexity of the assay systems are significantly reduced. For example, the absence of (i) a cell membrane structure which has the potential to create a thermodynamic sink for steroid hormone molecules and (ii) endogenous steroid hormone metabolism observed with cell based systems, provides for an assay system with enhanced sensitivity. Further, and advantageously, according to the test kits, assays and methods described herein, the relative amounts of essential structural elements (e.g. steroid hormone receptor and nucleic acid response element inclusive of one or more activated receptor binding domains) may be precisely controlled to provide enhanced assay functionality and increased sensitivity. Further still, the relative amounts of other co-factors including, for example, the steroid hormone receptor cofactor(s), transcription and/or translation machinery, as well as steroid metabolism machinery should also be precisely controlled to provide enhanced assay functionality and increased sensitivity. To further illustrate this point, reference is made to the experiments outlined in FIGS. 43 and 44. Initially Applicants demonstrated the importance of controlling the relative confluence/density of yeast cells in cell culture for Assay Prototype 0 (FIG. 43) where at higher cell densities (i.e. OD₆₀₀=0.4) reporter construct activity following the addition of testosterone could not be distinguished from a negative ethanol control; whereas at lower cell densities (i.e. OD₆₀₀=0.2) there was measurable difference between testosterone and control samples. These initial observations were extended to Assay Prototype 2 involving cell-free extracts (FIG. 44) where Applicants demonstrated that it was necessary to control the relative concentration of cell-free extract in order to specifically distinguish testosterone from the control. Again, these data highlight the importance of precisely controlling the relative amounts of the test kit/assay components in order to achieve detection of a ligand of interest.

To highlight the comparative sensitivity and molecular complexity, Table I below lists fundamental assay characteristics including sensitivity, molecular complexity, detection means, performance time etc for the Assay Prototypes according to the present invention (i.e. Assay Prototypes 1-3) as compared to a cell-based assay from Saccharomyces cerevisiae (i.e. Assay Prototype 0).

TABLE I
Assay Prototype Comparison

| | Assay | Assay | Assay | Assay |
|---|---|---|---|---|
| | Prototype 0 | Prototype 1 | Prototype 2 | Prototype 3 |
| Molecular | High | High | Moderate | Minimal |
| Complexity | | | | |
| Assay | Protein | Protein | RNA | Direct HR-L |
| Method | expression | expression | Transcription | binding |
| Assay | β- | Synthesised | Synthesised | HR-L-RE |
| Product | galactosidase | GFP | RNA | complex |
| Detection | Colorimetric | Fluorescence | RTqPCR | Gel |
| Method | | | Fluorescence | electrophoresis |
| | | | | Capacitance |
| | | | | SPR |
| Sensitivity | sub-μMol | sub-nMol | sub-pMol | sub-nMol |
| Performance | ~2 days | ~5hrs | ~2-5 hrs | <1hr |
| Time | | | | |
| (approx.) | | | | |

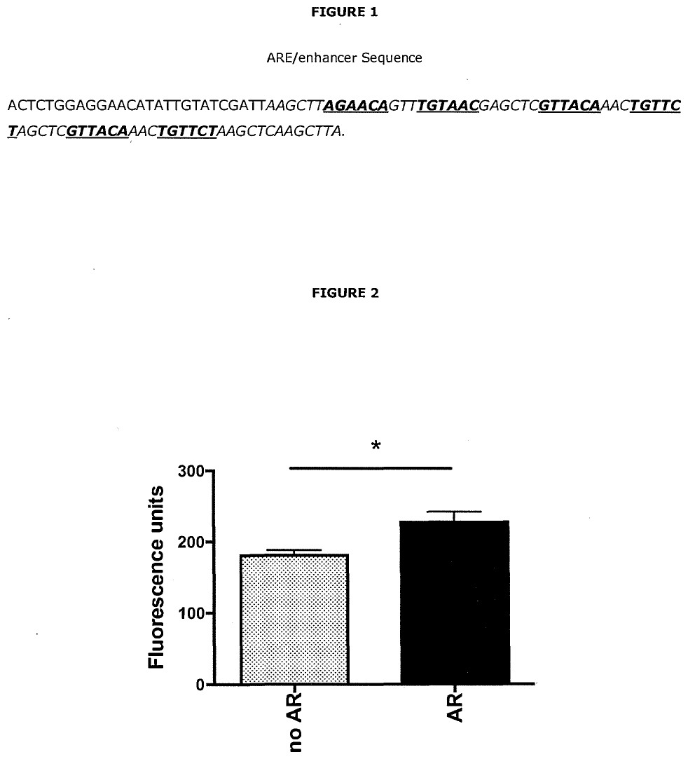

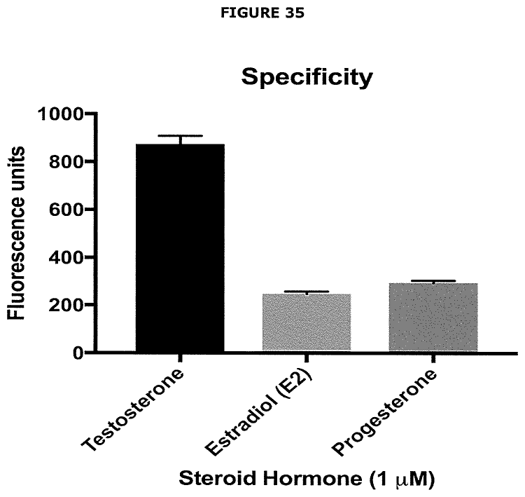

To further illustrate the relative sensitivity of the assays described herein, assay sensitivity was validated according to Examples 2 and 5 read in conjunction with FIGS. 1-6, 44 and 45. Briefly, an example Assay Prototype 1 was engineered using an androgen receptor and an androgen response element operably linked to a green fluorescent protein reporter. Binding between testosterone activated androgen receptor (i.e. a testosterone-androgen receptor complex) and an androgen receptor response element was measured by determining the level of GFP expression. With reference to Examples 2 and 5 and FIGS. 34 and 35, testosterone dose response curves were generated. The EC₅₀ for Prototype 1 (i.e. cell-free assay) was determined as 7.9×10⁻¹¹, which represents a ˜100-fold increase in sensitivity over Prototype 0 (i.e. cell-based assay) where published EC₅₀ values are in the order of 5×10⁻⁹ M (Death et al. (2004) J. Clin. Endo. Metabol. 89:2498-2500).

According to the test kits, assays and methods described herein, Applicants have determined the optimal amounts of steroid hormone receptor to nucleic acid response element should be x:1, where x is defined as [[0.2≤x≤20], which includes, but is not limited to, 0.2:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.75:1, 4:1, 4.25:1, 4.5:1, 4.75:1, 5:1, 5.5:1, 6:1, 6.5:1, 7:1, 7.5:1, 8:1, 9:1, 10:1, 11:1, 12:1, 13:1, 14:1, 15:1, 16:1, 17:1, 18:1, 19:1 and 20:1. The reason for this relates to the observation that in its ligand-bound/activated form, the steroid hormone receptor forms a dimer (e.g. (SR-L)₂) which then binds to and activates its associated response element. Accordingly, by precisely controlling the amounts of steroid hormone receptor relative to response element, the overall sensitivity of the assay is significantly enhanced since, in the presence of ligand, the assay will be configured to measure the optimum number of binding interactions. In contrast, it is difficult, if not impossible, to replicate the same degree of control for cell-based reporter assays which have been configured to detect the presence of a ligand, since the total copy number of the gene or nucleic acid sequence encoding (e.g.) recombinant receptor and/or nucleic acid response element cloned into the cell cannot be predicted or controlled with any accuracy.

Further, the data shown in FIG. 46 demonstrate the importance in controlling the relative amount of steroid hormone receptor cofactors in the assay. In particular, the relative levels of testosterone induced activation of the response element (as measured by total fluorescence) showed that too much HSP90 (i.e. 200 ng) inhibited fluorescence more than no HSP90 (i.e. 0 ng), whereas optimal signal was measured in the presence of 100 ng HSP90. These data further reinforce the importance in being able to precisely control assay parameters to achieve the optimal signal, and therefore assay sensitivity, for the detection of a ligand from a test sample.

While Applicants have determined that the optimal ratio of steroid hormone receptor to nucleic acid response element may optimally reside within the range ≥0.3:1 and ≤7:1, ratios outside of this range are also contemplated by the present invention. For example, where the ratio of steroid hormone receptor to nucleic acid response element is (e.g.) x:1, where x is the amount of steroid hormone receptor and x=0.2, 8, 9, 10, 11, 12, 13, 14, 15, 16, 17, 18, 19 and 20.

Further, while Applicants have determined that one optimal ratio of steroid hormone receptor to nucleic acid response element should be in the range ≥0.3:1 and ≤7:1, care must be taken not saturate the assay system with too much receptor, since this in itself may create thermodynamic or kinetic barriers that prevent optimal binding (e.g. a ratio of steroid hormone receptor to nucleic acid response element exceeding 20:1, and depending on assay conditions 7:1). However, a person of ordinary skill in the art could determine, as a matter of routine, the optimal sensitivity of an assay based on the relative amounts of its component parts.

Yet another advantage conferred by the test kits and assays according to the present invention is the relative ease of performance. In other words, performance of the test kits, assays and methods described herein does not require complex cell culture techniques, experienced laboratory technicians or convoluted laboratory testing equipment and analysis. This is particularly advantageous, because the test kits, assays and methods according to the present invention may be practiced by untrained personnel in the field following relatively simple testing procedures. Further, performance of the test kits, assays and methods may provide real time information (e.g.) when testing for performance enhancing substances in a sample taken from an athlete immediately prior to, or following, competition.

Accordingly, in an aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid response element

wherein, the presence of a ligand in the sample is determined when the sample is combined with the test kit and binding between the receptor-ligand complex and the nucleic acid sequence is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to this aspect of the present invention, the test kit further comprises a steroid hormone receptor cofactor that helps hold the steroid hormone receptor in an optimally folded and hormone responsive state (i.e. for ligand binding). Examples of steroid hormone receptor cofactors according to the present invention include, but are not limited to, heat shock protein 70, heat shock protein 40, heat shock protein 90, p23, heat shock protein organizing protein (Hop), 48 kD Hip protein, p60, and FKBP52.

In a related example according to this aspect of the present invention, the test kit comprises a heat shock protein (HSP). In a further related example, the HSP is HSP90.

In another example, the steroid hormone receptor cofactor is provided by a cell-free extract or is provided in a recombinant or synthetic form.

In a further example according to this aspect of the present invention, the nucleic acid response element comprises a promoter operably linked to a reporter construct. In yet a further example, the reporter construct comprises a sequence encoding at least one RNA aptamer, which following transcription, folds to form a structure capable of binding a fluorophore, the fluorescence of which is only activated or increased on binding to the RNA aptamer. In this way, binding of the fluorophore to the RNA aptamer generates a detectable fluorescent signal that reports the nucleic acid has been activated by a ligand-receptor complex, ultimately reflecting the presence of a ligand in the sample tested. Refer (e.g.) to Examples 4 and 7 read in conjunction with FIGS. 46 and 47, which follow.

In a further aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a steroid hormone receptor cofactor; and
(iii) a nucleic acid response element that is bound by the receptor-ligand complex; and
(iv) a detection means for detecting binding between the receptor-ligand complex and the nucleic acid response element

wherein, the presence of a ligand in the sample is determined when the sample is combined with the test kit and binding of the receptor-ligand complex to the nucleic acid sequence is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In a further aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a steroid hormone receptor cofactor; and
(iii) a nucleic acid response element that is bound by the receptor-ligand complex; and
(iv) a detection means for detecting binding between the receptor-ligand complex and the nucleic acid response element

wherein the relative amount of steroid hormone receptor to nucleic acid response element in the test kit is x:1, where x is the amount of steroid hormone receptor and is defined as [0.2≤x≤20], and

wherein, the presence of a ligand in the sample is determined when the sample is combined with the test kit and binding of the receptor-ligand complex to the nucleic acid sequence is detected.

In an example, the relative amount of steroid hormone receptor to nucleic acid response element in the test kit is x:1, where x is the amount of steroid hormone receptor and is defined as [0.3≤x≤7].

The test kits as described herein may further comprise steroid metabolism machinery to facilitate detection of intrinsically inactive ligands.

As such, in a further aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a steroid hormone receptor cofactor and/or steroid metabolism machinery; and
(iii) a nucleic acid response element that is bound by the receptor-ligand complex; and
(iv) a detection means for detecting binding between the receptor-ligand complex and the nucleic acid response element

wherein, the presence of a ligand in the sample is determined when the sample is combined with the test kit and binding between the receptor-ligand complex and the nucleic acid sequence is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

The test kits as described herein may further comprise a cell-free extract.

As such, in yet a further aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a steroid hormone receptor cofactor and/or steroid metabolism machinery and/or a cell-free extract; and
(iii) a nucleic acid response element that is bound by the receptor-ligand complex; and
(iv) a detection means for detecting binding between the receptor-ligand complex and the nucleic acid response element

wherein, the presence of a ligand in the sample is determined when the sample is combined with the test kit and binding of the receptor-ligand complex to the nucleic acid sequence is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In another example according to these and other aspects of the present invention, the nucleic acid response element is linked to a reporter construct, and binding of the receptor-ligand complex to the nucleic acid response element is determined by interrogating transcription or translation of the reporter construct.

As such, the test kits described herein further comprise transcription and/or translation machinery to achieve successful transcription and/or translation of the reporter construct.

In an example, the transcription and/or translation machinery is provided by a nuclear or cell-free extract.

In another example, the reporter construct comprises a promoter sequence and a reporter construct, and the promoter sequence is activated when the nucleic acid response element is bound by the receptor-ligand complex. Examples of promoters include, but are not limited to, minimal CMV, minimal pA promoters, minimal TKHSV promoters, minimal TATA-like promoters.

In a further example, the nucleic acid response element and the promoter driving expression of the reporter construct are operably linked.

In a related example, the nucleic acid response element and the reporter construct are comprised within the same, or on different, nucleic acid sequences.

In another related example, the nucleic acid response element, promoter and enhancer sequence and reporter construct are comprised within the same, or on different, nucleic acid sequences, including examples of where certain elements are comprised within the same nucleic acid sequence while others are comprised within separate nucleic acid sequences.

As such, the test kits, assays and methods according to the present invention may be configured to detect transcript levels of a reporter construct by investigating, for example, messenger ribose nucleic acid (mRNA) or complementary deoxyribose nucleic acid (cDNA) levels when a test sample is combined with the assay, as a means to screen the sample for the presence of a ligand having steroid hormone receptor activity.

The present invention further contemplates a reporter construct comprising one or more copies of an RNA aptamer sequence which, when transcribed, folds to form a structure capable of binding a fluorophore, the fluorescence of which is only unlocked/activated on binding to the RNA aptamer. Examples of such RNA aptamer/fluorophore combinations include, without limitation, Spinach and its associated fluorophore 3,5-difluoro-4-hydroxybenzylidene imidazolinone (DFHBI); Spinach2 and its associated fluorophores DFHBI-1T and DFHBI-2T; iSpinach and its associated fluorophores DFHBI-1T and DFHBI-2T; Broccoli and its associated fluorophore DFHBI-1T; Corn and its associated fluorophore DFHO0; Mango and its associated fluorophores derived from thiazole orange (TO; including TO-1 and TO-3); DiR2s-Apt and its associated fluorophore OTB; AptII-mini3-4c and its associated fluorophore Hoescht; DNB and its associated fluorophore RG-DN; BHQ apt(A1) and its associated fluorophore CY3-BHQ1; Red-Broccoli and its associated fluorophore DFHO; DNB and its associated fluorophores TMR-DN and SR-DN; DIR aptamer and its associated fluorophore DIR; MG aptamer and its associated fluorophore Mal Green; DIR2s-Apt and its associated fluorophore DIR-pro; SRB apt and its associated fluorophore Patent Blue (refer (e.g.) to Bouhedda F. Et al., International Journal of Molecular Sciences, 2017). Importantly, it is the binding interaction between the fluorophore and the RNA aptamer that potentiates fluorescence of the molecule, notwithstanding that some fluorophores may have low level innate or background fluorescence.

Accordingly, in another aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a reporter construct operably linked to the nucleic acid response element,

wherein, the reporter construct is activated when the receptor-ligand complex binds to the nucleic acid response element, and

wherein, the presence of a ligand in the sample is determined when the sample is combined with the test kit and transcription of the reporter construct is detected.

In a further aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a reporter operably construct linked to the nucleic acid response element; and
(iv) transcription machinery

wherein, the reporter construct is activated when the receptor-ligand complex binds to the nucleic acid response element, and

wherein, the presence of a ligand in the sample is determined when the sample is combined with the test kit and transcription of the reporter construct is detected.

According to this aspect of the present invention the test kit may further comprise a steroid hormone receptor cofactor, steroid metabolism machinery and/or a cell-free extract as described herein.

In certain examples according to this aspect of the present invention, reporter gene transcript levels comprising (e.g.) mRNA or cDNA may be semi-/quantified using established techniques such as quantitative polymerase chain reaction (qPCR, including RealTime qPCR and Reverse Transcription-qPCR), or using other techniques such as fluorescence based on detection of intercalating dyes. For example, the use of fluorophores that bind to RNA aptamers to form RNA-fluorophore complexes, as described herein. These and other techniques would be known to a person skilled in the art.

Accordingly, in another aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex; and
(ii) a nucleic acid response element that is bound by the receptor-ligand complex; and
(iii) a reporter construct operably linked to the nucleic acid response element, wherein the reporter construct comprises at least one RNA aptamer sequence capable of binding to a fluorophore

wherein, the reporter construct is activated when the receptor-ligand complex binds to the nucleic acid response element, and

wherein, the presence of a ligand in the sample is determined when the sample is combined with the test kit and fluorescence activated by binding of the fluorophore to at least one RNA aptamer is detected.

In an example according to this aspect of the present invention, the nucleic acid response element and the reporter construct are comprised on the same nucleic acid sequence. In a related example, the nucleic acid sequence comprising the nucleic acid response element and the reporter construct is defined by SEQ ID NO: 19 as follows:

SEQ ID NO: 19
TGGAGAACAGCCTGTTCTCCATCTAGATGGAGAACAGCCTGTTCTCCATC
TAGATGGAGAACAGCCTGTTCTCCATCTAGAGCCGCCCCGACTGCATCTG
CGTGTTCGAATTCGCCAATGACAAGACGCTGGGCGGGGTTTGTGTCATCA
TAGAACTAAAGACATGCAAATATATTTCTTCCGGGGACACCGCCAGCAAA
CGCGAGCAACGGGCCACGGGGATGAAGCAGAAGCTTCGAATCGCGAATTC
GCCCACCATGGGGAGACAGCCTACGAGCCTGAGCCTCCAGTCTTGCCATG
TGTATGTGGGTACGAAGGAGAGGAGAGGAAGAGGAGAGTACCCACATACT
CTGATGATCCTTCGGGATCATTCATGGCAA

In yet another example according to this aspect of the present invention, the test kit further comprises a detection means for detecting transcription of the reporter construct.

In an example according to this aspect of the present invention, the test kit further comprises transcription machinery to facilitate transcription of the reporter construct comprising RNA aptamer.

In another example according to this aspect of the present invention, the RNA aptamer is selected from Spinach, iSpinach and Broccoli, and the fluorophore which binds to the RNA aptamer thereby generating a fluorescent signal is 3,5-difluoro-4-hydroxybenzylidene imidazolinone (DFHBI).

In another example according to this aspect of the present invention, the RNA aptamer is Mango, and the fluorophore which binds to the RNA aptamer thereby generating a fluorescent signal is thiazole orange (TO).

In a further example according to these and other aspects of the present invention, the reporter construct comprises a single sequence copy of the RNA aptamer, or multiple sequence copies of the RNA aptamer, (e.g.) two, three, four, five, six etc copies of the sequence encoding the RNA aptamer. A person skilled in the art will recognize that the copy number of RNA aptamer sequences will be governed by the optimal signal to noise ratio, as determine by routine optimization studies. In a further example, reporter construct comprises four copies of the RNA aptamer sequence iSpinach, sometimes referred to in the art as ‘4×Spinach’. The DNA sequence encoding the RNA aptamer iSpinach (SEQ ID NO: 17) is as follows:

SEQ ID NO: 17
AGGAGTACGGTGAGGGTCGGGTCCAGTAGGTACGCCTACTGTTGAGTAGA
GTGTGGGCTCCGTACTCCC

A DNA template sequence for 4×Spinach, used to engineer a reporter construct/assay prototype reported in Example 7, is well documented in the art.

Alternatively, the assay may be configured to detect protein expression levels of a reporter construct when a test sample is combined with the assay, as a means to screen the sample for the presence of a ligand having the ability to bind to a steroid hormone receptor and elicit a genomic response in a cell.

Accordingly, in yet another aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a reporter construct operably linked to the nucleic acid sequence

wherein, the reporter construct is activated when the receptor-ligand complex binds to the nucleic acid response element, and

wherein, the presence of a ligand in the sample is determined when the test sample is combined with the test kit and translation of the reporter construct is detected.

In a further aspect of the present invention there is provided a test kit for screening a test sample for the presence of a ligand, which ligand is capable of forming a complex with a steroid hormone receptor and eliciting a genomic response when in a cell, the test kit comprising:
(i) a hormone receptor that forms a receptor-ligand complex with a ligand from the test sample; and
(ii) a nucleic acid response element that is bound by the receptor-ligand complex; and
(iii) a reporter construct operably linked to the nucleic acid sequence; and
(iv) transcription and translation machinery

wherein, the reporter construct is activated when the receptor-ligand complex binds to the nucleic acid response element, and

wherein, the presence of a ligand in the sample is determined when the sample is combined with the test kit and translation of the reporter construct is detected.

According to this aspect of the present invention the test kit may further comprise a steroid hormone receptor cofactor and/or steroid metabolism machinery and/or a cell-free extract as described herein.

Again, detecting gene expression levels based on translation of the reporter construct will depend on the nature of the reporter marker used. For example, the reporter construct may include a gene encoding a fluorescent protein such as green fluorescent protein (GFP), red fluorescent protein (RFP), orange fluorescent protein etc, and the fluorescent gene product could be detected and semi-/quantified using spectral based methods known to a person skilled in the art. Alternatively, there is widely established methodology around detection of (e.g.) β-galactosidase (LacZ) and β-glucuronidase (GUS), luciferase, and so reporter constructs for use in the test kits, assays and methods according to the present invention may be engineered to include genes encoding these gene products.

Examples 2 and 5, read in conjunction with FIGS. 1-6, 33 and 34, describes a test kit/assay according to this aspect of the present invention.

In yet another aspect of the present invention there is provided a test kit for for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid response element

wherein, the presence of a ligand in the sample is determined when the sample is combined with the test kit and binding between the receptor-ligand complex and the nucleic acid response element is detected.

According to this aspect of the present invention the test kit may further comprise a steroid hormone receptor cofactor and/or steroid metabolism machinery and/or transcription and/or translation machinery and/or a cell-free extract as described herein.

Also according to this aspect of the present invention, the binding interaction between the nucleic acid response element and the activated hormone receptor/receptor-ligand complex is measured directly. A direct binding interaction may be detected using any number of techniques, including without limitation, optical methods, spectroscopy, visible spectroscopy, Raman spectroscopy, UV spectroscopy, surface plasmon resonance, electrochemical methods, impedance, resistance, capacitance, mechanical sensing by changes in mass, changes in mechanical resonance, electrophoresis, gel electrophoresis, gel retardation, imaging, fluorescence and fluorescence resonance energy transfer.

In a further example of the present invention, the hormone receptor is selected from an androgen receptor, an estrogen receptor including estrogen receptor alpha (ER-α) and estrogen receptor beta (ER-β), a progesterone receptor including progesterone receptor A (PRA) and progesterone receptor B (PRB), a mineralocorticoid receptor, and a glucocorticoid receptor.

As such, in other aspects of the present invention there is provided a test kit for screening a test sample for the presence of a ligand, which ligand is capable of forming a complex with a steroid hormone receptor and eliciting a genomic response when in a cell, the test kit comprising:
(i) an androgen receptor that forms a receptor-ligand complex with a ligand from the test sample; or
(ii) an estrogen receptor that forms a receptor-ligand complex with a ligand from the test sample, wherein the estrogen receptor is estrogen receptor alpha or estrogen receptor beta; or
(iii) a progesterone receptor that forms a receptor-ligand complex with a ligand from the test sample, wherein the progesterone receptor is progesterone receptor A or progesterone receptor B; or
(iv) a mineralocorticoid receptor that forms a receptor-ligand complex with a ligand from the test sample; or
(v) a glucocorticoid receptor that forms a receptor-ligand complex with a ligand from the test sample; and
(vi) a nucleic acid response element that is bound by the receptor-ligand complex as defined in any one of (i) to (v) above; and
(vii) a detection means for detecting binding between the receptor-ligand complex and the nucleic acid response element

wherein, the presence of a ligand in the test sample is determined when the sample is combined with the test kit and binding between the receptor-ligand complex and the nucleic acid sequence is detected.

In a further aspect of the present invention there is provided a test kit for screening a test sample for the presence of a ligand, which ligand is capable of forming a complex with a steroid hormone receptor and eliciting a genomic response when in a cell, the test kit comprising:
(i) an androgen receptor that forms a receptor-ligand complex with a ligand from the test sample; or
(ii) an estrogen receptor that forms a receptor-ligand complex with a ligand from the test sample, wherein the estrogen receptor is estrogen receptor alpha or estrogen receptor beta; or
(iii) a progesterone receptor that forms a receptor-ligand complex with a ligand from the test sample, wherein the progesterone receptor is progesterone receptor A or progesterone receptor B; or
(iv) a mineralocorticoid receptor that forms a receptor-ligand complex with a ligand from the test sample; or
(v) a glucocorticoid receptor that forms a receptor-ligand complex with a ligand from the test sample; and
(vi) a nucleic acid response element that is bound by the receptor-ligand complex as defined in any one of (i) to (v) above; and
(vii) a detection means for detecting binding between the receptor-ligand complex and the nucleic acid response element; and
(viii) a steroid hormone receptor cofactor, steroid metabolism machinery, transcription and/or translation machinery and/or a cell-free extract as described herein

wherein, the presence of a ligand in the test sample is determined when the sample is combined with the test kit and binding between the receptor-ligand complex and the nucleic acid sequence is detected.

In an example according to these aspects of the present invention, the nucleic acid response element is selected from an androgen response element, an estrogen response element, a progesterone response element, a mineralocorticoid response element or a glucocorticoid response element, depending on the nature of the sample to be tested, and likely population of ligands to be interrogated.

In another example, the androgen response element comprises a DNA binding motif that selectively binds to an activated androgen receptor. In a related example, the DNA binding motif binds to a dimer of the ligand bound androgen receptor (i.e. (AR-L)₂; where “AR” is an androgen receptor and “L” is a ligand). In a further related example, the DNA binding motif contains a dihexameric palindrome to create binding specificity between the activated androgen receptor and associated response element.

In yet another example, the estrogen response element comprises a DNA binding motif that selectively binds to an activated estrogen receptor. In a related example, the DNA binding motif binds to a dimer of the ligand bound estrogen receptor (i.e. (ER-L)₂; where “ER” is an estrogen receptor selected from ER-α or ER-β). In a further related example, the DNA binding motif contains a dihexameric palindrome to create binding specificity between the activated estrogen receptor and associated response element.

In a further example, the progesterone response element comprises a DNA binding motif that selectively binds to an activated progesterone receptor. In a related example, the DNA binding motif binds to a dimer of the ligand bound progesterone receptor (i.e. (PR-L)₂; where “PR” is an estrogen receptor selected from PRA or PRB). In a further related example, the DNA binding motif contains a dihexameric palindrome to create binding specificity between the activated progesterone receptor and associated response element.

In yet a further example, the mineralocorticoid response element comprises a DNA binding motif that selectively binds to an activated mineralocorticoid receptor. In a related example, the DNA binding motif binds to a dimer of the ligand bound mineralcorticoid receptor (i.e. (MR-L)₂; where “MR” is a mineralocorticoid receptor). In a further related example, the DNA binding motif contains a dihexameric palindrome to create binding specificity between the activated mineralocorticoid receptor and associated response element.

In yet another example, the glucocorticoid response element comprises a DNA binding motif that selectively binds to an activated glucocorticoid receptor. In a related example, the DNA binding motif binds to a dimer of the ligand bound glucocorticoid receptor (i.e. (GR-L)₂; where “GR” is a glucocorticoid receptor). In a further related example, the DNA binding motif contains a dihexameric palindrome to create binding specificity between the activated glucocorticoid receptor and associated response element.

In yet another example according to these and other aspects of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

Accordingly, in yet a further aspect of the present invention there is provided a test kit for screening a test sample for the presence of a ligand, which ligand is capable of forming a complex with a steroid hormone receptor and eliciting a genomic response when in a cell, the test kit comprising:
(i) a hormone receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid response element

wherein the relative amount of steroid hormone receptor to nucleic acid response element in the test kit is x:1, where x is the amount of steroid hormone receptor and is defined as [0.2≤x≤20], and

wherein, the presence of a ligand in the sample is determined when the sample is combined with the test kit and binding between the receptor-ligand complex and the nucleic acid response is detected.

In an example according to this aspect of the present invention, the relative amount of steroid hormone receptor to nucleic acid response element in the test kit is x:1, where x is the amount of steroid hormone receptor and is defined as [0.3≤x≤7].

According to this aspect of the present invention the test kit may further comprise a steroid hormone receptor cofactor, steroid metabolism machinery, transcription and/or translation machinery and/or a cell-free extract as described herein.

Although the assays according to the present invention describe systems in which the response elements comprise palindrome sequences that only recognize, and selectively bind to dimeric, hormone receptor-ligand complexes (i.e. (HR-L)₂), the present invention further contemplates other types of protein/nucleic acid binding interactions between activated hormone receptors and response elements. For example, non-(HR-L)₂ complexes such as (HR-L)₁, (HR-L)₃ may be configured to bind to various response elements or, depending on the assay design and functionality, activated hormone receptors where there are two ligands bound to one receptor (e.g. HR-(L)₂), or multiple receptors bound to a single ligand (e.g. (HR)₂-L) or multiple receptors bound to multiple ligands (e.g. (HR)₃-(L)₂) etc, all of which may selectively bind to a DNA motif within the response element and activate a genomic response based on the promoter/enhancer architecture.

A person skilled in the art will appreciate that the test kits, assays and methods of the present invention may include one or more receptor types that, upon activation via ligand binding, are capable of forming (e.g.) homodimers or heterodimers (or indeed any other ligand-receptor complex architecture). By way of illustration only, in the case of detection of estrogen and estrogen-like steroid, or non-steroid, hormones, the test kits, assays and methods may comprise a mixture of both estrogen receptor alpha (ER-α) and estrogen receptor beta (ER-β) receptors that, upon activation by a steroid ligand such as estradiol, may form (e.g.) a homodimer of ER-α or ER-β (i.e. (ER-α)-(ER-α); or (ER-β)-(ER-β)) or a heterodimer of ER-α and ER-β (i.e. (ER-α)-(ER-β); or (ER-β)-(ER-α)). Similarly, in the case of detection of progestin and progestin-like steroid, or non-steroid, hormones, the test kits, assays and methods may comprise a mixture of both progesterone receptor A (PRA) and progesterone receptor B (PRB) that upon activation by a steroid ligand such as a progestin may form (e.g.) a homodimer of PRA or PRB (i.e. PRA-PRA or PRB-PRB), or a heterodimer of PRA and PRB (i.e. PRA-PRB or PRB-PRA).

In another example of the present invention, the steroid hormone receptor is purified from a cell, or is derived from a cell-based hormone receptor through recombinant cloning, expression and purification.

In yet another example of the present invention, the hormone receptor is synthetic, and its synthesis is either (i) modeled on an endogenous cell-based hormone receptor or (ii) modeled on an engineered form of an endogenous cell-based hormone receptor where the receptor has been engineered (e.g.) to improve the binding kinetics of the ligand for its receptor to improve assay sensitivity, or (e.g.), to engineer the receptor with a peptide handle for immobilization to a substrate or solid support to assist with performance of the assay. By way of illustration only, a person skilled in the art will understand that an androgen receptor has five different domains (nominally A/B, C, D, E and F domains), and any one of these domains may be mutated in order to engineer the receptor to render it more or less sensitive to ligand binding. The structure of other hormone receptors would be known to a person skilled in the relevant art of steroid hormone detection, and could be similarly engineered.

A person skilled in the art would recognize that modifications to steroid hormone receptors (or any other test kit/assay components) such as those described immediately above may be engineered via synthetic and recombinant means.

A person skilled in the art would also recognize that any steroid hormone receptor may be employed in the test kits, assays and methods of the present invention, provided that it retains the ability to bind to, and be activated by, a ligand of interest for detection. This includes, steroid hormone receptors, based on endogenous cellular forms, as well as recombinant forms and includes recombinant hormone receptors engineered for increased, or decreased, ligand affinity.

As such, the test kits, assays and methods according to the present invention may be configured to screen/detect any ligand that elicits a steroid response. However, a person skilled in the art will recognise that, according to the various assays concepts described herein, detection of different hormone classes (i.e. ligands) requires the format of the test kits, assays and methods must be properly configured and optimized. For example, detection of a ligand that binds to and activates an androgen receptor, such as testosterone as well as other testosterone-like hormones, requires test kits/assays comprising androgen receptor together with an androgen response element capable of binding to an activated androgen-receptor complex.

In an example of the present invention, the androgen response element is a nucleic acid sequence comprising one or more sequences set forth in:

### SEQ ID NO: 1

### Androgen Response Element; Sense Strand

GGTACAnnnTGTTCT where n is any nucleotide; or

### SEQ ID NO: 2

### Androgen Response Element; Antisense Strand

CCATGTnnnACAAGA where n is any nucleotide.

Similarly, detection of a ligand that binds to and activates an estrogen receptor, such as estradiol, estrone, other estrogen-like steroid hormones and non-steroidal estrogen receptor modulators, requires requires test kits/assays comprising either an estrogen receptor alpha (ER-α) or estrogen receptor beta (ER-β) together with an estrogen response element capable of binding to an activated estrogen receptor complex.

In another example of the present invention, the estrogen response element is a nucleic acid sequence comprising one or more sequences set forth in:

### SEQ ID NO: 3

### ER-α Response Element; Sense Strand

AGGTCAnnnTGACCT where n is any nucleotide; or

### SEQ ID NO: 4

### ER-α Response Element; Antisense Strand

TCCAGTnnnACTGGA where n is any nucleotide; or

### SEQ ID NO: 5

### ER-β Response Element; Sense Strand

AGGTCAnnnTGACCT where n is any nucleotide; or

### SEQ ID NO: 6

### ER-β Response Element; Antisense Strand

TCCAGTnnnACTGGA where n is any nucleotide.

Similarly, detection of a ligand that binds to and activates a progesterone receptor, such as progestogens and other progestogen-like hormones, requires test kits/assays comprising an progesterone receptor A (PRA) or progesterone receptor B (PRB) together with a nucleic acid based progesterone response element capable of binding to an activated progesterone receptor.

In yet another example of the present invention, the progesterone response element is a nucleic acid sequence comprising one or more sequences set forth in:

### SEQ ID NO: 7

### PRA Response Element; Sense Strand

AGAACAnnnTGTTCT where n is any nucleotide; or

### SEQ ID NO: 8

### PRA Response Element; Antisense Strand

TCTTGTnnnACAAGA where n is any nucleotide.

### SEQ ID NO: 9

### PRB Response Element; Sense Strand

AGAACAnnnTGTTCT where n is any nucleotide; or

### SEQ ID NO: 10

### PRB Response Element; Antisense Strand

TCTTGTnnnACAAGA where n is any nucleotide.

Similarly, detection of a ligand that binds to and activates a mineralocorticoid receptor, such as aldosterone and other aldosterone-like hormones or non-steroidal mineralocorticoid receptor modulators, requires test kits/assays comprising a mineralocorticoid receptor together with a nucleic acid based mineralocorticoid response element capable of binding to an activated mineralocorticoid receptor.

In yet another example of the present invention, the mineralocorticoid response element is a nucleic acid sequence comprising one or more sequences set forth in:

### SEQ ID NO: 11

### Mineralocorticoid Response Element; Sense Strand

AGAACAnnnTGTTCT where n is any nucleotide; or

### SEQ ID NO: 12

### Mineralocorticoid Response Element; Antisense Strand

TCTTGTnnnACAAGA where n is any nucleotide.

Similarly, detection of a ligand that binds to and activates a glucocorticoid receptor, such as cortisol, other cortisol-like hormones and non-steroidal glucocorticoid receptor modulators, requires test kits/assays comprising a glucocorticoid receptor together with a nucleic acid based glucocorticoid response element capable of binding to an activated glucocorticoid receptor.

In yet another example of the present invention, the glucocorticoid response element is a nucleic acid sequence comprising one or more sequences set forth in:

### SEQ ID NO: 13

### Glucocorticoid Response Element; Sense Strand

AGAACAnnnTGTTCT where n is any nucleotide; or

### SEQ ID NO: 14

### Glucocorticoid Response Element; Antisense Strand

TCTTGTnnnACAAGA where n is any nucleotide.

As previously stated, the various response elements incorporate binding motifs configured to selectively bind activated ligand-receptor complexes. For example, each of the androgen, estrogen, progesterone, mineralocorticoid and glucocorticoid response elements comprise palindromic dihexameric sequences which in their secondary structure orientations facilitate binding of dimerized ligand receptor complex (i.e. (HR-L)₂) to the response element.

The test kits, assays and methods described herein may further comprise translation and/or transcription machinery, one or more steroid hormone receptor cofactors, including but not limited to heat shock proteins, and/or buffers so as to enhance the functionality and/or sensitivity of the test kits, assays and methods described herein.

In an example, the steroid hormone receptor cofactor according to the present invention includes, but is not limited to, heat shock protein 70, heat shock protein 40, heat shock protein 90, p23, heat shock protein organizing protein (Hop), 48 kD Hip protein, p60, and FKBP52.

The present invention further contemplates detection of one or more physiologically inactivate ligands from a test sample, which ligands are ultimately capable of activating steroid hormone receptors when converted to a physiologically active form. As such, the test kits, assays and methods as described herein further comprise steroid metabolism machinery that is capable of processing the ligand in such a way that it will activate its corresponding steroid hormone receptor. In this way, detection of physiologically inactive ligands (e.g. prohormones) from samples such as nutritional supplements is possible.

Accordingly, in yet a further aspect of the present invention there is provided a test kit for screening a test sample for the presence of a ligand, which ligand is capable of forming a complex with a steroid hormone receptor and eliciting a genomic response when in a cell, the test kit comprising:
(i) a steroid hormone receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid response element; and
(iv) a steroid hormone receptor cofactor, transcription and/or translation machinery, steroid metabolism machinery and/or a cell-free extract

wherein, the presence of a ligand in the sample is determined when the sample is combined with the test kit and binding between the receptor-ligand complex and the nucleic acid sequence is detected.

In an example according to this aspect of the present invention, the relative amount of steroid hormone receptor to nucleic acid response element in the test kit is x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

In certain examples, the steroid hormone receptor cofactor, translation and/or transcription machinery and/or steroid metabolism machinery may be derived from one or more cell-free extracts as defined herein.

In another example, the test sample is a biological sample. In a related example, the biological sample is a body fluid sample, including but not limited to, blood, plasma, serum, saliva, interstitial fluid, semen and urine.

In another example, the test sample is derived from an animal including, but not limited to, an equine animal, a canine animal, a dromedary animal, a bovine animal, a porcine animal, an ovine animal, a caprine animal, an avian animal, a simian animal, a murine animal, a leporine animal, a cervine animal, a piscine animal, a salmonid animal, a primate animal, a simian animal, and a human animal.

In another example, the test sample is a non-biological sample. In a related example, the non-biological sample includes, but is not limited to, a liquid sample including water, a soil sample, a textile sample including but not limited to plastics, a mineral sample, a food sample and a medication.

Examples of a food sample includes, but is not limited to, vegetables, meats, beverages, supplements and herbal extracts.

Examples of a medication includes, but is not limited to, drugs, tonics, syrups, pills, lozenges, creams, sprays and gels.

Designer steroids and non-steroidal anabolic drugs pose a significant and growing challenge for anti-doping laboratories. First identified in the early 2000s with the detection of tetrahydrogestrinone and madol, the threat posed by designer anabolic drugs has rapidly increased to include numerous potential agents. These synthetically-derived anabolic drugs are designed to evade detection or legal controls with respect to both manufacture and supply, and many are widely available on the internet where they are sold as so-called “supplements”.

Mass spectrometry remains the primary technology for the identification of known illicit steroid hormones and non-steroid anabolic drugs in biological samples and/or supplements. Despite its sensitivity and specificity, mass spectrometry remains limited by requiring prior knowledge of the steroid and non-steroid anabolic drug's chemical structures for detection. Moreover, mass spectrometry fails to provide information about the biological activity of the anabolic drugs detected, and is unable to differentiate between bioactive and inactive molecules. This is information that is required for legal prosecution of athletes, coaches, trainers, managers and manufacturers.

In recent years, yeast and mammalian cell-based in vitro androgen bioassays have been used to detect the presence of novel synthetic androgens, the androgenic potential of progestins as well as androgens, pro-androgens, designer androgens and designer non-steroid anabolic drugs in supplements. However, these assays suffer limitations associated with molecular complexity, as described elsewhere herein, and require technical skills that are both molecular and microbiological in nature, are time consuming, labour intensive and expensive. As such, it is not feasible to consider the assays in their present form for inclusion in routine screening. In other words, yeast and mammalian cell-based assays suffer significant limitations because not high throughput or cost effective.

Advantageously, the present invention provides activity based test kits, assays and methods that work fundamentally on the principle of steroid hormone receptor activation. By detecting steroid hormone receptor activation by a ligand present within a sample to be tested, the present invention provides cell-free test kits, assays and methods that do not rely on structural knowledge of the ligand(s) being interrogated, can readily distinguish between the presence of biologically active and inactive ligands, and provide cost-effective, reliable and reproducible systems that do not require complex laboratory equipment or particular expertise to perform.

Accordingly, in an example according to the test kits, assays and methods described herein, the ligand is a performance enhancing designer drug and/or steroid.

In another example according to the test kits, assays and methods described herein, the ligand is of an unknown chemical structure.

In a further example according to the test kits, assays and methods described herein, the ligand is of a previously unknown chemical structure.

The present invention also contemplates assay methods based on the test kits described herein.

Accordingly, in yet another aspect of the present invention there is provided an assay method for screening a test sample for the presence of a ligand, which ligand is capable of forming a complex with a steroid hormone receptor and eliciting a genomic response when in a cell, the assay method comprising the steps of:

(i) providing assay reagents comprising:
(a) a steroid hormone receptor that forms a receptor-ligand complex with a ligand from the test sample; and
(b) a nucleic acid sequence that is bound by the receptor-ligand complex; and
(c) a detection means for detecting binding between the receptor-ligand complex and the nucleic acid sequence; and

(ii) combining the test sample with the assay reagents

wherein, the presence of a ligand in the sample is determined when the test sample is combined with the test kit and binding between the receptor-ligand complex and the nucleic acid sequence is detected.

According to this aspect of the present invention, the relative amount of steroid hormone receptor to nucleic acid response element in the test kit may be x:1, where x is the amount of steroid hormone receptor and is defined as [0.2≤x≤20]. In a related example, the relative amount of steroid hormone receptor to nucleic acid response element in the test kit is x:1, where x is the amount of steroid hormone receptor and is defined as [0.3≤x≤7].

According to this aspect of the present invention the test kit may further comprise a steroid hormone receptor cofactor, steroid metabolism machinery, transcription and/or translation machinery and/or a cell-free extract as described herein.

According to the methods described herein, the test result may be compared to a reference threshold in order to determine the absolute level of signal generated by a ligand present in a test sample. Indeed, Applicants observed non-specific binding and/or activation of the response element by non-ligand bound receptor (e.g. FIG. 32). Accordingly, where it is desirable to perform a semi-quantitative analysis for any given test sample, the assays and methods described herein may be performed in the absence of test sample to first establish a reference threshold (e.g. in presence of ethanol acting as a negative control). Assay results obtained from a test sample may be then be compared to the reference threshold, to determine the absolute activity attributable to the ligand(s) present in a sample using a simple subtraction methodology.

The present invention further contemplates the use of the assays and test kits described herein to determine the potency of a test compound relative to a reference compound. According to the present invention, the term ‘relative potency’ is defined as the multiplier of biological activity of a test compound relative to a reference compound, as determined by normalizing the biological activity of the test compound to the reference compound.

The biological activity of the test and reference compounds may be determined using EC₅₀ or the concentration of compound that gives half the maximal response from a dose response curve for that particular compound. The dose response curve is generated by serially diluting the compound and measuring its steroid hormone receptor binding/activation profile. A plot of the measured activity (e.g. as measured by fluorescence, absorbance, chemiluminescence etc) vs concentration of the compound (i.e. serial dilution of the compound generates a concentration range that is best presented on a log scale) is then made.

To further illustrate the concept of relative potency, an example measurement and calculation of relative potency is presented in FIG. 44. The androgen receptor binding/activation activity of testosterone (T; reference) was compared to dihydrotestosterone (DHT; test compound), where the measured EC₅₀ values were 4.08×10⁻⁹ (T) and 1.74×10⁻⁹ (DHT). The relative potency was determined by normalizing the EC₅₀ value for T to the EC₅₀ value for DHT (i.e. 4.08×10⁻⁹/1.74×10⁻⁹) to yield a relative potency of ˜2.3. In other words, in this experiment, dihydrotestosterone was 2.3 times more potent in binding to and activating its target androgen receptor than testosterone.

A person skilled in the art will recognize that a measure of relative potency of a test compound is relative to the reference compound used. In other words, the relative potency of a test compound is likely to differ depending on the reference compound to which its biological activity is normalized.

Where the relative potency is >1, the test compound invokes a higher measured biological activity in the assays compared to the reference compound. Where the relative potency is <1, the test compound invokes a lower measured biological activity in the assays compared to the reference compound. Where the relative potency=1, the test compound and the reference compound invoke equal biological activity in the assays.

Relative potency can also be used to determine the activation factor of a test compound in question. As used herein, activation factor relates the relative potency of a test compound determined in a yeast cell or using a yeast cell-free extract (i.e. which contains no metabolic machinery) to the relative potency of the same test compound determined in a mammalian cell or using a mammalian cell-free extract (i.e. which includes metabolic machinery) as a measure of relative activation between the two states of the test compound. An activation factor >1 means that the test compound has undergone metabolic conversion to a more physiologically active state in the presence of metabolic machinery in the assay.

To further illustrate this point Applicant determined the EC₅₀ and relative potency of a known androgen anabolic steroid ‘Jungle Warfare’ to dihydrotestosterone in both a yeast and human cell line in Table II as follows:

TABLE II
Relative Potency and Activation Factor for Jungle
Warfare and BMS-564929 (known SARM)

| | Yeast | Relative | HuH7 | Relative | Activation |
|---|---|---|---|---|---|
| | EC₅₀ | Potency | EC₅₀ | Potency | Factor |
| Supplement | (nM) | [to DHT] | (nM) | [to DHT] | (AF) |
| Jungle Warfare | 9.82 | 0.25 | 0.73 | 10 | 40 |
| BMS-564929 | 0.004157 | 4.787×10⁻⁴ | 0.4653 | 0.607 | 1268 |

The activation factor was calculated by dividing the relative potency measured in a mammalian cell (i.e. inclusive of metabolic machinery capable of converting the androgen from a physiologically inactive to a physiologically active form, from a less physiologically active to a more physiologically active form, from a more physiologically active form to a less physiologically active form, or from a physiologically active form to a physiologically inactive form) to the relative potency measured in a yeast cell (i.e. without metabolic machinery). This yielded an AF of ˜40 (i.e. AF=RP (mammalian)/RP (yeast)), which in this example and taking into account the EC₅₀ values, means ‘Jungle Warfare’ exists as a physiologically active supplement which, following metabolism, converts to a form that is more physiologically active than the DHT, being the reference compound against which its activity has been normalized for the purpose of these calculations.

In yet another example, Applicant determined the EC₅₀ and relative potency of a known selective androgen receptor molecule (SARM) with a designated classification of BMS-564929. This SARM has been previously detected in both race horses and humans. As determined by its relative potency in yeast relative to DHT (i.e. no metabolic machinery), BMS-546929 had a very low relative potency value approximating 5×10⁻⁴ (Table II). However, when the EC₅₀ of BMS-546929 was measured in HuH7 cells where it is metabolised, it becomes a much more potent andorgen with a RP of ˜60% (i.e. 0.607) to that of DHT. Consequently, its Activation Factor of >1200 reflects that BMS-546929 is converted from an inactive selective androgen receptor modulator to an active selective androgen receptor modulator following metabolism.

These data highlight the activating power of metabolism when interrogating certain compounds/ligands, and the importance of an assay system optionally inclusive of metabolic machinery capable of metabolising a compound/ligand derived from a sample under interrogation (i.e. converting a compound/ligand from a physiologically inactive form to a physiologically active form, from a physiologically active form to a more physiologically active form, from a more pysiologically active form to a less physiologically active form, or from a phsyiologically active form to a physiologically inactive form). In the absence of this feature, the assay systems may not always be reliable in detecting prodrug/non-metabolised forms of (e.g.) designer drugs and the like, which might otherwise evade detection.

In yet another aspect of the present invention there is provided an assay method for screening a test sample for the presence of a ligand, which ligand is capable of forming a complex with a steroid hormone receptor and eliciting a genomic response when in a cell, the assay method comprising the steps of:

(i) providing assay reagents comprising:
a. a steroid hormone receptor that forms a receptor-ligand complex with a ligand from the test sample; and
b. a nucleic acid response element that is bound by the receptor-ligand; and
c. a detection means for detecting binding between the receptor-ligand complex and the nucleic acid response element; and

(ii) combining the test sample with the assay reagents

wherein the relative amount of steroid hormone receptor to nucleic acid response element in the test kit is x:1, where x is the amount of steroid hormone receptor and is defined as [0.2≤x≤20], and

wherein the presence of a ligand in the sample is determined when the sample is combined with the test kit and binding between the receptor-ligand complex and the nucleic acid response element is detected.

In an example, the relative amount of steroid hormone receptor to nucleic acid response element in the test kit is x:1, where x is the amount of steroid hormone receptor and is defined as [0.3≤x≤7].

In certain examples, the inventions described herein have utility in the detection of performance enhancing pro/drugs (e.g. anabolic steroids and selective androgen receptor modulators) used in human as well as non-human athletes including race horses, camels and dogs.

Accordingly, in another aspect of the present invention there is provided a method for determining the doping status of an athlete, the method comprising combining a test sample obtained from the athlete with a test kit as described herein.

In an example according to this aspect of the present invention, the test sample obtained from the athlete is a serum or plasma sample.

In a further aspect of the present invention there is provided an article of manufacture for screening a test sample for the presence of a ligand, which ligand is capable of activating a steroid hormone receptor and eliciting a genomic response in a cell, the article of manufacture comprising a test kit as described herein together with instructions for how to detect the presence of a ligand in the sample.

In yet a further aspect of the present invention there is provided an article of manufacture for determining doping in an athlete, the article of manufacture comprising a test kit as described herein together with instructions for detecting the presence of a ligand in a sample derived from the athlete, wherein the presence of the ligand in the sample is indicative of doping in the athlete.

Finally, the present invention further contemplates use of the test kits, assays and methods as described herein for detecting antagonists of a target ligand by screening a sample of interest for a compound that will prevent binding of the ligand to its steroid hormone receptor such that it no longer activates the receptor and elicits a genomic response.

This is particularly useful when there is a need to screen for antagonists that block steroid hormone receptor activation (e.g.) as potential therapeutics for the treatment of endocrine and non-endocrine cancers. For example, the activity based test kits, methods and assays comprising one or more estrogen receptors according to the present invention can be used to screen compound libraries for the presence of antagonists of estrogen receptor activation in breast cancer tissue.

The invention is further described with reference to the following examples. It will be appreciated that the invention as claimed is not intended to be limited in any way by these examples.

### EXAMPLES

The information and data which follows demonstrates various prototype assays with respect to the detection of ligands that bind to and activate the androgen receptor, including (e.g.) testosterone and dihydrotestosterone. These Examples are used to illustrate the activity assay platform described and claimed herein, where the assay concepts and principles exemplified by the detection of androgen ligands would apply equally to the detection of other receptor ligands of interest including, without limitation, ligands that bind to estrogen receptor including estradiol, ligands that bind to progesterone receptor including progesterone, ligands that bind to mineralocorticoid receptor, and ligands that bind to the glucocorticoid receptor.

### Example 1

### Assay Concept Overview

### 1.1 IVT Platform

The main components of the IVT platform includes an androgen response element (ARE) enhancer-driven minimal promoter DNA template, recombinant androgen receptor (AR), a cell- or nuclear-extract that contains transcription-ready machinery, and a transcription or transcription/translation buffer that allows for RNA synthesis, or both RNA and protein synthesis. The minimal promoter will drive a basal level of RNA molecule synthesis and subsequent protein. Activation of transcription by ligand-activated AR bound to the ARE/enhancer will increase the amount of RNA molecule synthesis and protein levels.

### Androgen Response Element (ARE)

The androgen response elements that were tested in these experiments include:
1. Mouse mammary tumor virus (MMTV), a response element that is strongly transcribed in response to steroid hormones
2. Enhancer/ARE—this is more specific to AR because of the enhancer region. The consensus ARE DNA sequence shows high homology to the response elements of glucocorticoid receptor (GR), mineralocorticoid receptor (MR) and progesterone receptor (PR). To increase the ARE specificity to androgens an adjacent androgen-specific enhancer region was used.

### Androgen Receptor (AR)

Commercial recombinant androgen receptor (AR) was tested from independent commercial sources including AR obtained from both Abcam and Sigma-Aldrich.

### Cell or Nuclear Extract

The cell- or nuclear extracts that were tested include commercial HeLa (cervical cancer cell line) extract, in-house produced HeLa cell extract, in-house produced PC-3 (prostate cancer cell line) extract, in-house produced HEK293 (kidney) extract and in-house produced HuH7 (human liver cell line) extract.

### 1.2 Description of ARE-Mediated Transcription/Translation

Natural androgen signaling starts with the diffusion of an androgenic molecule into the cell where it binds to the AR being held in an inactive state in the cytoplasm, bound to heat shock protein 90 (HSP90). Upon binding the androgenic molecule (or ligand), AR undergoes a conformational change that releases HSP90, and exposes nuclear localization and dimerization sites. The ligand-AR complex translocates to the nucleus where AR binds to ARE sites in the DNA and instructs RNA polymerase II to initiate transcription of AR-regulated genes. Transcription of a gene by RNA polymerase II produces an mRNA transcript that, in turn, acts as the template for the ribosome machinery to make a protein.

In simplified terms, Androgen-AR binds to DNA [Step 1; Assay Prototype 3 in Example 4] to produce mRNA [Step 2; Assay Prototype 2 in Example 3] that acts as a template for protein synthesis [Step 3; Assay Prototype 1 in Example 2].

### Example 2

### Assay Prototype 1: Are/Enhancer-Regulated Reporter Protein Synthesis

### 2.1 Overview

After AR binds to the ARE/enhancer, it orchestrates RNA polymerase II to interact with the cellular transcription machinery to produce a messenger RNA molecule that will, in turn, act as a template for protein synthesis. The end result is synthesis of new protein.

In these experiments, the ARE/enhancer sequence was cloned upstream of the pA-MinProm-Green Fluorescence Protein (GFP) sequence in plasmid pSF-pA-minPromGFP (Oxford Genetics) such that when introduced into a coupled in vitro transcription/translation reaction, this DNA template would drive ligand-activated AR to generate GFP.

### 2.2 Approach and Results

(1) The ARE/enhancer sequence was designed to have an enhancer sequence followed by a 3× tandem ARE sequence, as shown in FIG. 1 (SEQ ID NO: 15).

Sense and antisense oligonucleotides were commercially synthesized by Sigma-Aldrich as separate molecules. The separate strands were annealed by a routine temperature hybridisation reaction. Annealing was confirmed by 3% gel electrophoresis. The synthesized ARE/enhancer sequence incorporated restriction enzyme SalI- and SmaI-overhang sequences at the 5′ and 3′ ends, respectively, so directed restriction enzyme cloning could be used to insert the ARE/enhancer sequence DNA into plasmid pSF-pA-minPromGFP, also digested with SalI and SmaI. The plasmid DNA was then linearized with restriction enzyme PvuI before use in the in vitro transcription/translation reaction.

(2) A commercially available in vitro transcription/translation kit (ThermoFisher Scientific, 1-step human coupled IVT kit) was used. The kit contains HeLa cell extract, accessory proteins, and reaction mix. To the reaction mix, recombinant AR protein was added (Abcam).
(3) To initiate transcription, 4 nM testosterone (T) was added to the assembled reactions. For the negative control, AR was not added to the reactions. The reactions were incubated at 30° C., with shaking 300 rpm, for 2 h.

The results are presented in FIG. 2 where testosterone-activated AR induces transcription and translation of GFP.

Next, the inducibility of the assay was determined. The reactions were set up as above, with the exception that 50 μl reactions were prepared. The no AR negative control to measure (baseline promoter activation) was included as well as a no DNA template control (to measure auto-fluorescence of the cell lysate). To activate transcription/translation, 4 nM testosterone was added, or as a non-activated vehicle control, ethanol was added at final concentration 0.1% v/v. Reactions were incubated at 30° C. with shaking at 300 rpm for 5 h.

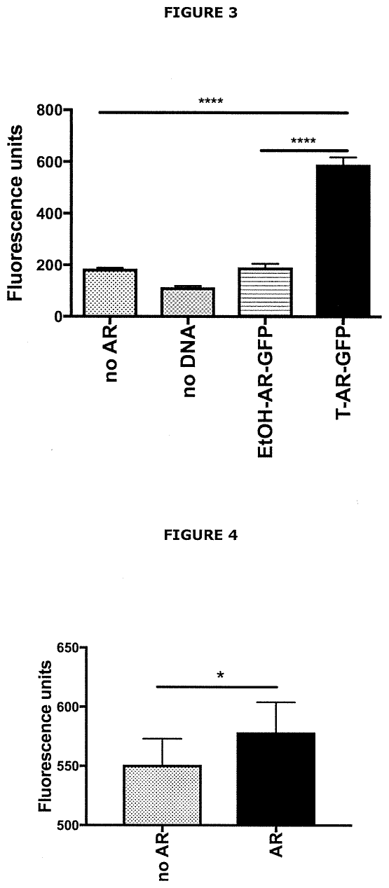

The results are shown in FIG. 3, where the level of fluorescence, as generated by translation of Green Fluorescent Protein, was significantly higher in the testosterone induced AR activation reaction compared to the various control reactions.

Ideally, one application of the assay would be the ability to use the testing in field applications (e.g. racehorse or athletes trackside). To this end, Applicant tested whether freeze-drying reactions onto the surface of filter paper discs led to viable in vitro transcription/translation reactions.

Reactions (50 μl) were assembled as above (test and no AR as the negative control) and then pipetted onto small circular discs of Whatman filter paper. Immediately after transfer of the reaction mix, the filter paper was snap frozen in liquid nitrogen and transferred to a freeze dryer, after which the reaction-paper discs were stored at −80° C. overnight.

The following day the paper discs were reconstituted in nuclease-free water, and brought up to 30° C. temperature. The in vitro transcription/translation reaction was initiated with 4 nM testosterone and the reaction mixtures incubated at 30° C. with shaking at 300 rpm, for 6 h.

The results are shown in FIG. 4 where AR-positive assay reactions had increased fluorescence compared to the no AR control. The fluorescence high baseline for the no AR reaction is mostly likely due to the autofluorescence of the paper discs.

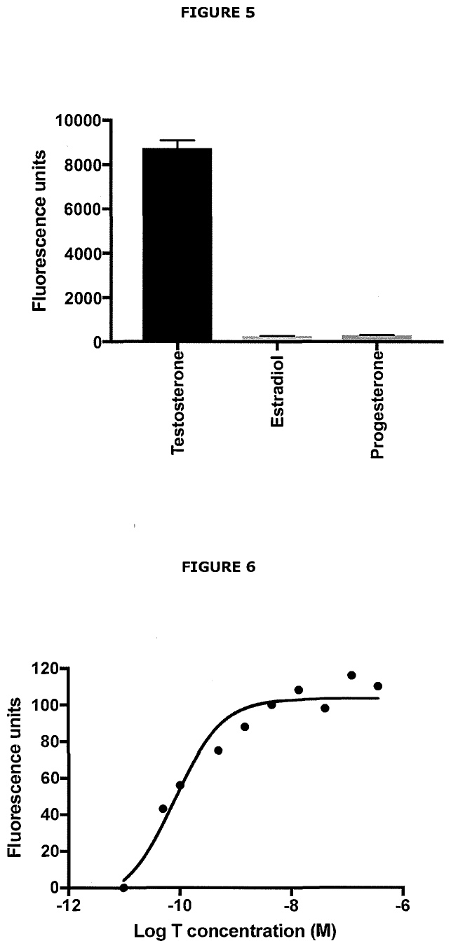

To demonstrate assay specificity, Applicant showed testosterone-specific activation by demonstrating non-responsiveness to other sex hormones (e.g.) estradiol and progesterone. These data are shown in FIG. 5, where even at high dose (i.e. μM concentration), estradiol and progesterone were unable to activate the AR-ARE/enhancer-regulated in vitro transcription/translation assay. These data also demonstrate that increased testosterone [μM versus nM (FIG. 3)] induced higher levels of fluorescence, indicative of more AR being produced with higher testosterone concentrations, as would be expected (fluorescence units 8000 vs 600, respectively).

The sensitivity of the assay was next tested. In vitro transcription and translation reactions were assembled, and activated by testosterone across the concentration range of 3.7×10⁻⁷ to 5×10⁻¹¹M. The data are shown in FIG. 6 where the approximate EC₅₀ (dose that leads to half maximal response) is 7.9×10⁻¹¹M.

In summary, in vitro coupled transcription/translation assay (i.e. Assay Prototype 1) successfully generated GFP from an ARE/enhancer-regulated DNA template, following AR activation by testosterone. These data show Assay Prototype 1 is (i) specific to testosterone, and is unable to be activated by estradiol or progesterone, (ii) is sensitive across the physiological range, nM to subnM, and (iii) the reaction mixture is able to be snap frozen, freeze dried and reconstituted after storage at −80° C., a finding that exemplifies important first steps for a field test version of this test.

### Example 3

### Assay Prototype 2: ARE/Enhancer-Regulated RNA Synthesis

A number of different approaches were investigated for detecting and measuring the amount of RNA molecules generated from an in vitro transcription (IVT, no longer coupled to translation) reaction, including:
(i) direct detection of RNA molecules (i.e.) proof of principle
(ii) direct detection of the RNA molecule through incorporation of fluorophore-labelled NTPs
(iii) RTqPCR
(iv) RNA aptamer and fluorophore binding

As there was no longer the need to produce a protein from the mRNA transcript, the mRNA transcript used in this assay prototype validation was: (a) a truncated GFP sequence since shorter molecules are more protected from degradation; (b) a synthetic mRNA sequence; or (c) a RNA aptamer sequence.

### 3.1 Prototype 2 Direct Detection of RNA

### (i) Visualization of IVT-Generated RNA Molecules

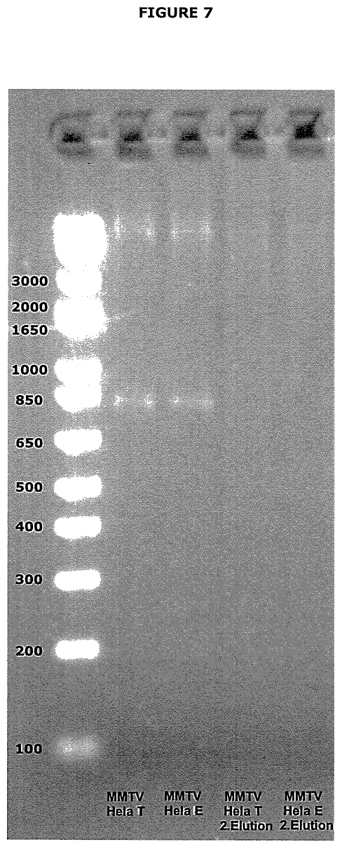

To show that the IVT reactions produced RNA that could be quantified by cyanine-5-NTP labelling, RTqPCR or RNA aptamer detection, the IVT reactions were prepared with the MMTV-luciferase DNA template and activated with testosterone (4 nM) or as vehicle control, ethanol (0.1% v/v). After a 1 h incubation, the RNA/DNA was purified from the transcription reactions by standard column purification and then the RNA/DNA molecules were subjected to agarose gel electrophoresis. DNA and RNA molecules were then visualized by SYBR green dye. The results are presented in FIG. 7 where an 850 bp RNA band, being the expected size of the transcript, was detected.

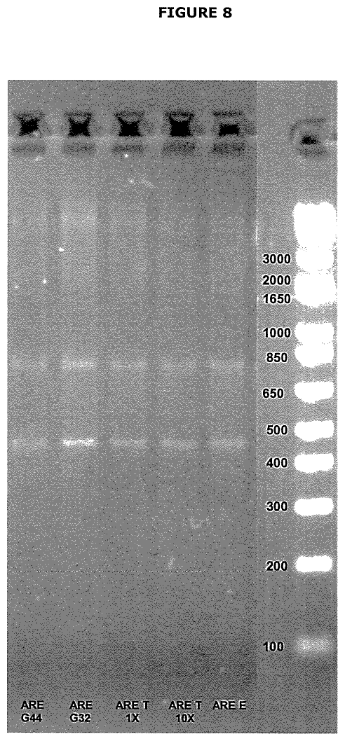

Similarly, to show that ARE/enhancer IVT reactions were producing RNA molecules, IVT reactions were prepared with the ARE/enhancer DNA template and activated with testosterone (4 nM 1× T or 40 nM 10× T), ethanol (0.1% v/v), G32 or G44 (equine plasma samples from gelding racehorses). After a 1 h incubation, the RNA/DNA was purified from the transcription reactions by standard column purification and then the RNA/DNA molecules were subjected to agarose gel electrophoresis. DNA and RNA were visualized with SYBR green dye. The data are presented in FIG. 8.

### (ii) Fluorescent Detection of RNA Molecules Produced in the IVT Reaction

Next, the RNA molecules produced in the IVT reaction were quantified using the Quant-IT RNA assay (Molecular Probes Life Technologies). The assay is based on a fluorescent dye that is highly selective for RNA over double-stranded DNA. After the dye binds to RNA, the complex is stable for 3 hours. IVT reactions were prepared, with the ARE/enhancer DNA template, and stimulated with testosterone (4 nM), and incubated at 30° C. for 1 h. No NTP reactions acted as controls.

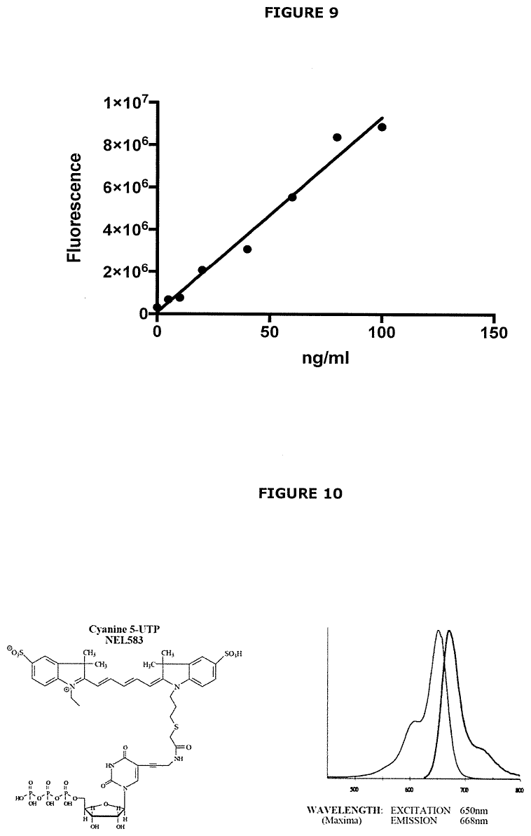

The results are shown in FIG. 9, and Table III as follows:

TABLE III
RNA molecule detection by Quant-IT RNA assay.

| | |
|---|---|
| IVT reaction 19.06.18 ARE-T | 168ng/ml |
| (replicate #1) | |
| IVT reaction 19.06.18 ARE-T | 153ng/ml |
| (replicate #2) | |
| IVT reaction 19.06.18 ARE-T | 143ng/ml |
| (replicate #3) | |
| IVT reaction 19.06.18 no NTP | 22ng/ml |
| (control for DNA) | |

These data show that there is approximately 7-times more RNA transcript in AR-activated reactions than control reactions.

### 3.2: Direct Detection of RNA Through Incorporation of Fluorophore-Labelled NTPs

The next approach developed for detecting the RNA molecules generated in the IVT reactions involved incorporating fluorescently-labelled NTPs into the RNA molecule. The RNA molecules are synthesized by RNA polymerase II which adds one NTP (UTP, CTP, ATP, GTP) at a time, with the NTPs representing the building blocks of the RNA molecule. The idea underlying this approach was to substitute UTP for cyanine-5-labelled UTP (cy-5-UTP) or CTP for cyanine-5-labelled CTP (cy-5-CTP) or both. The cyanine-5-labelled UTP (cy-5-UTP) and associated wavelength excitation and emission spectra is shown in FIG. 10.

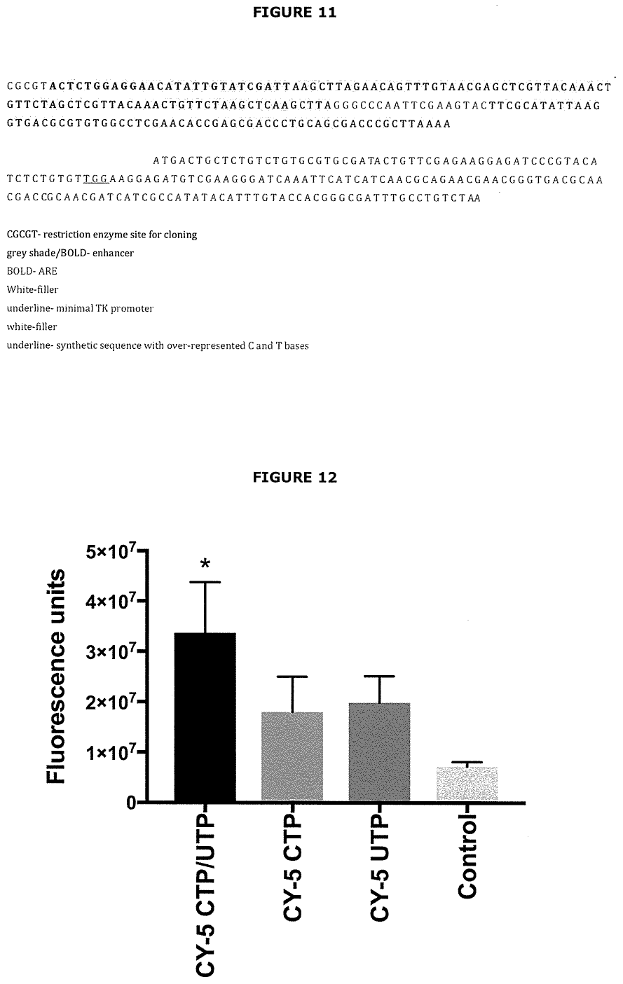

For these experiments the truncated ARE/enhancer GFP DNA sequence was switched out for a synthetic DNA sequence optimized for UTP and CTP, as shown in FIG. 11. This template would increase the incorporation of labelled-UTP and/or labelled-CTP. The sequence designed and cloned into the ARE/enhancer/minTKpromoter plasmid is shown below (note it is shown as the DNA template, not the RNA, where dTTP in DNA is a code for UTP in RNA). This synthetic DNA does not encode for a gene, and would not produce a valid messenger RNA and therefore, protein.

The DNA was synthesized commercially (Sigma-Aldrich) as a short DNA molecule and provided in plasmid pA. Escherichia coli competent cells were transformed with the plasmid (selected with ampicillin resistance), and the E. coli cells used to propagate the plasmid DNA. The plasmid DNA was isolated from bacterial cultures and used as a template for standard PCR. The PCR-amplified DNA molecule was then purified using phenol/chloroform extraction followed by ethanol precipitation, before it was used as the DNA template (470 bp) in the IVT reactions. The IVT reactions were activated by testosterone (4 nM), where UTP was replaced with a cy-UTP:UTP combination, or CTP was replaced with a cy-5-CTP:CTP combination, or both UTP and CTP were replaced with a cy-5-UTP:UTP:cy-5-CTP:CTP combination. Column purification was used to purify the RNA molecule from the transcription reaction (to remove free cy-NTP) and resuspended in 100 μL nuclease-free water. The flurophore was excited at 630 nm and fluorescence emission was measured at 650 nm. For some reactions, the MMTV-luciferase was used as the DNA template to exemplify both AR DNA binding sites. The results are shown in FIG. 12.

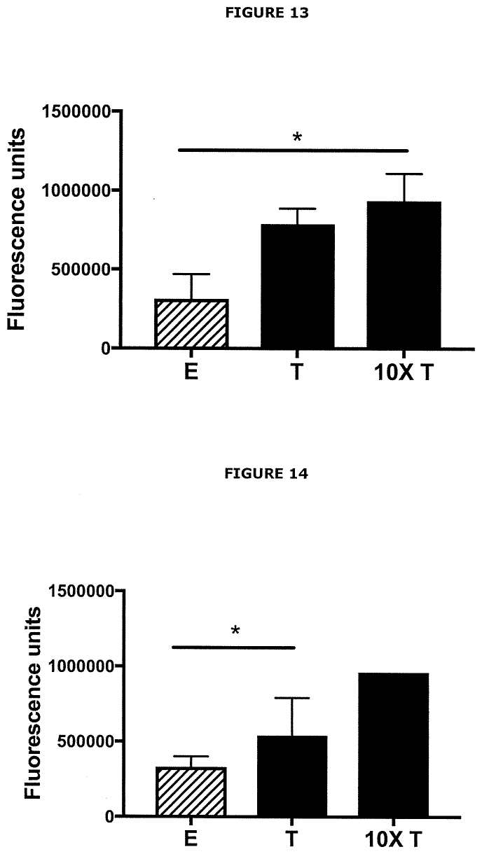

Next the level of activation by testosterone versus its inactive vehicle control (ethanol) was tested for both the ARE/enhancer synthetic- and MMTV-luciferase DNA templates. IVT reactions were assembled with the cy-5-UTP:UTP:cy-5-CTP:CTP combination and either the ARE/enhancer synthetic DNA template or MMTV-luciferase DNA template. The reactions were activated with testosterone (4 nM) or its inactive control (0.1% (v/v) ethanol). The results are presented in FIG. 13 and FIG. 14. Comparing the fluorescence readings for ARE/enhancer synthetic DNA template versus the MMTV-luciferase DNA template, the readings for 4 nM testosterone were higher for the synthetic DNA template. This was in keeping with the higher number of U and C bases that were engineered into the synthetic DNA sequence to increase incorporation of the cy-5-labelled nucleotides (FIG. 11).

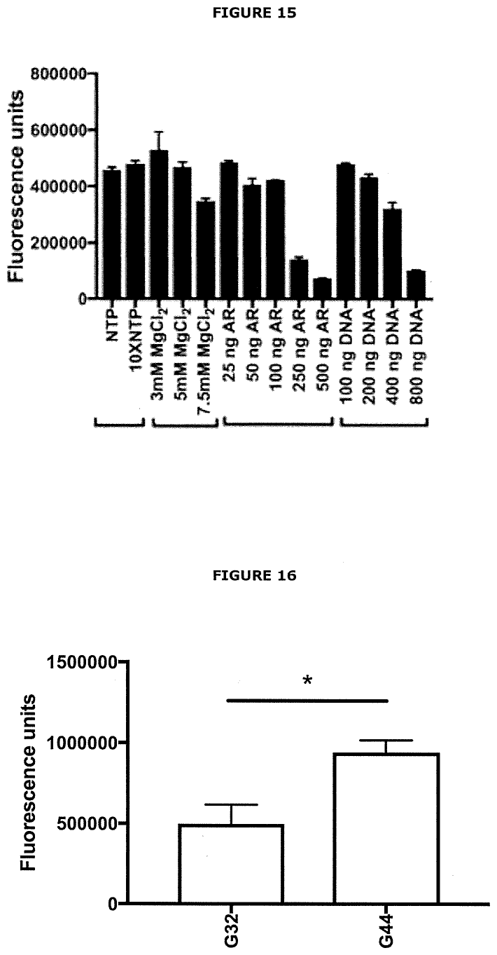

The IVT reaction contains core components AR, DNA template, MgCl₂ and NTPs. The next experiment tested whether changing the concentration of these core components of the IVT reaction affected output levels of the RNA molecule. As shown in FIG. 15, IVT reactions were prepared with 1× or 10×NTPs; 3 mM, 5 mM and 7.5 mM MgCl₂; 25 ng, 50 ng, 100 ng 250 ng, or 500 ng AR; 100 ng, 200 ng, 400 ng, or 800 ng DNA template. These data show that RNA molecule synthesis decreases with increasing concentrations of MgCl₂, AR and DNA template. Findings show that a range of AR to DNA templated to more RNA molecule synthesis. These data show that the optimal molar ratio of AR to DNA template lies in the range ≥0.3:1 and ≤7:1.

The next experiment investigated if the cy-5-NTP labelled approach could detect the difference between AR bioactivity in two equine plasma samples obtained from gelding racehorses, assigned G32 and G44. These two equine plasma samples have previously been tested with the HEK293-cell based AR bioactivity assay and G44 was shown to be more active than G32. IVT reactions were assembled with the cy-5-UTP:UTP or cy-5-CTP:CTP combination and the ARE/enhancer synthetic DNA template. The reactions were activated with gelding plasma (15% v/v). The results are presented in FIG. 16, and confirm that Assay Prototype 2 was able to correctly distinguish between the two field samples based on previous validation of its respective androgen content.

### 3.3 RNA Detection by RTqPCR (Version 1—MMTV-minProm-Truncated Luciferase Gene as DNA Template)

The first experiment for this assay prototype exemplified the MMTV-minProm-truncated luciferase gene DNA template, for IVT-RTqPCR measurements.

IVT reactions were assembled and activated with the addition of testosterone (4 nM) and then incubated at 30° C. for 2 h.

Following the IVT reaction, the DNA template was removed by DNaseI digestion (Baseline-Zero DNase, Epicentre). The RNA was purified by standard phenol/chloroform extraction followed by ethanol precipitation (or column purification). A standard reverse transcription reaction (Superscript VILO cDNA synthesis kit, ThermoFisher Scientific) was completed to generate cDNA. The cDNA was amplified by qPCR (KAPA SYBR Fast qPCR Kit) using a specific primer kit.

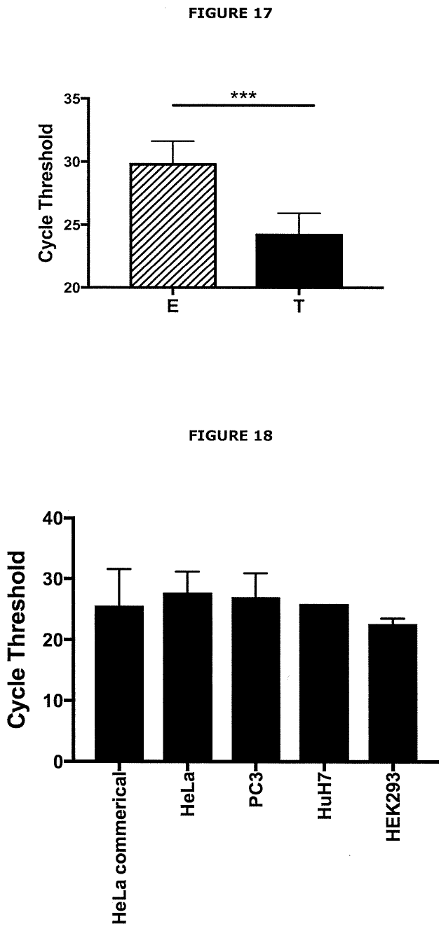

These data are presented in FIG. 17, which shows the raw data for cycle threshold as measured for RNA levels in ethanol-treated versus T-treated IVT reactions. PCR works by exponentially amplifying very small numbers of cDNA (=RNA) molecules to very large numbers of double stranded DNA molecules. If there are more starting cDNA molecules at the beginning of amplification then fluorescence detection of the double stranded DNA produced from those cDNA molecules will occur at a lower amplification cycle number, referred to as cycle threshold. In FIG. 17, the average cycle threshold number for testosterone is ˜24, while for ethanol it is ˜30. This suggests there was a 64-fold difference between starting RNA levels.

IVT/RTqPCR reactions with the MMTV-luciferase DNA template were next used to exemplify nuclear extracts prepared from different cell types. The nuclear extract that was primarily used for exemplification of the IVT reaction was HeLa nuclear extract (a cervical cancer cell line). This cell line does not express AR. This nuclear extract is commercially available (eg. Promega). In Applicant's laboratory, a protocol was established to make in-house HeLa nuclear extract. The same protocol was then used to prepare nuclear extracts from PC-3 (human prostate cancer cell line), HuH7 (human liver cell line), and HEK293 (human kidney cell line) cells. These data are presented in FIG. 18, and show that in-house nuclear extracts, from a variety of cell types, provide adequate transcriptional machinery to support IVT reactions. The cycle threshold data indicates that no extract led to significantly increased amounts of RNA transcription (indicated by a decrease in cycle threshold) or a marked decrease it output (indicated by an increase in cycle threshold).

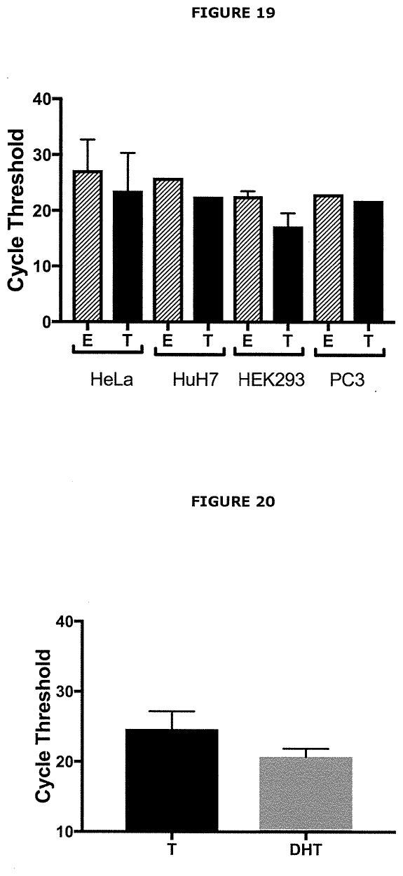

Next, the level of intrinsic activation of the different nuclear extracts was determined. IVT reactions were prepared as above with different nuclear extracts driving transcription, and reactions were activated with testosterone or with ethanol as vehicle control. These data are presented in FIG. 19 and show that all four cell lines produce transcriptionally active nuclear extracts that respond to testosterone. The HuH7 and HEK293 cells are stable cells lines expressing AR, PC3 cells express endogenous AR, while HeLa cells do not express AR.

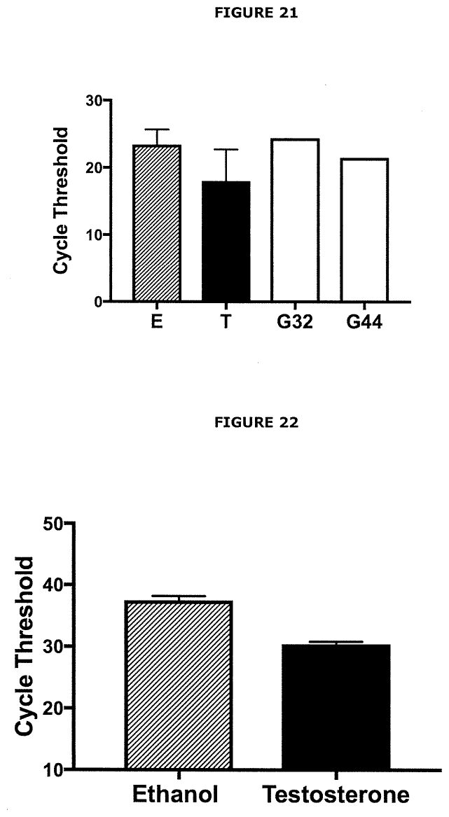

The IVT reactions exemplified thus far have only been activated with the major male endogenous androgen in males, namely testosterone. AR is activated by other natural androgens, such as dihydrotestosterone, a potent endogenous androgen that shows 4-times higher bioactivity for AR than testosterone. IVT reactions were prepared with the commercially-sourced HeLa nuclear extract and activated with either testosterone (T, 4 nM) or dihydrotestosterone (DHT, 4 nM). The MMTV-luciferase DNA template was used, with RTqPCR the readout for mRNA levels. These data are presented in FIG. 20, and show that DHT induced more RNA synthesis than T, as demonstrated by the lower cycle threshold (DHT˜20 compared to T ˜24). These results are in keeping with the reported higher binding affinity of AR for DHT, and its intrinsically higher activation of the AR.

### 3.4 RNA Detection by RTqPCR (Version 2—ARE/Enhancer-minProm-Truncated GFP Gene as DNA Template)

In this series of experiments the ARE/enhancer minProm GFP DNA template for IVT-RTqPCR measurements was exemplified.

During the development of version 2 (ARE/enhancer-minProm-GFP) a number of improvements were made that included addressing complete removal of the DNA template from the IVT reaction post-incubation and protecting the newly synthesised RNA from degradation. A subsequent development was to design a specific purpose-built primer set to ensure that the DNA template could not be amplified during the real-time PCR reaction.

To increase the destruction of the DNA template post IVT reaction, EDTA was removed from the IVT transcription buffer, as EDTA can inhibit DNase I. Removing EDTA had no effect on the IVT reaction (data not shown). Next the DNA was modified so that it could be immobilized on magnetic beads. PCR primers with a biotin group attached to the 5′-sense primer were used to amplify the DNA template. Using a 3′-antisense primer that truncated the GFP gene also allowed for the generation of a shorter DNA template, and therefore shorter mRNA transcript that is more resistant to degradation. The biotin-tagged DNA was then mixed with streptavidin-coated magnetic beads (Dynabeads 280) to capture the DNA on the beads. This improved DNA template removal because after the IVT reaction was complete, the DNA template could be separated from the IVT reaction through magnets, bringing the beads to the bottom of the tube. The IVT reaction was transferred to a fresh tube, and treated with ZeroBaseline DNase. This two-step approach decreased risk of residual DNA template entering the PCR reaction.

To test the modified IVT-RTqPCR system involving the DNA template immobilized on magnetic beads, IVT reactions were assembled as follows:

Reaction mix:

| | |
|---|---|
| Buffer + beads | 6 μl |
| MgCl₂ | 1.5 μl |
| AR (25ng) | 0.5 μl |
| NTP | 1 μl |
| dH20 | 10.23 μl |
| E/T | 1 μl |
| HeLa extract (8 units) | 3.8 μl |
| RNAse out | 0.5 μl |

The IVT reaction was activated by the addition of testosterone (4 nM), or ethanol (0.1% v/v) and incubated for 30° C. for 2 h. Equine samples, G32 and G44, were also tested in this modified system. Magnetic separation was used to separate the IVT reaction from the DNA template, then RNA purified by standard phenol/chloroform extraction followed by ethanol precipitation. Alternatively, RNA was purified by column purification. Epicentre Baseline Zero DNase I was then used to destroy any residual DNA template, before RT-qPCR was performed using a specific primer/probe set. The results are presented in FIG. 21 and show that testosterone (T) induced the generation of more RNA transcripts than ethanol (E). The results also demonstrate that the AR-IVT reaction was able to differentiate endogenous androgenic bioactivity in equine plasma samples, with G44 showing higher activity than G32.

To overcome the potential contamination of PCR reagents with DNA template, a revised reverse transcription step and PCR was designed. Using a specific reverse transcription primer, a stem loop structure was added to the end of the DNA template during the cDNA synthesis step. During the denaturation step of PCR, the stem loop structure opens up, exposing a PCR specific primer site. Using the specific reverse transcription primer followed by a specific reverse PCR primer, there can be no amplification of the original DNA template.

The IVT reaction is performed then phenol/chloroform extraction followed by ethanol precipitation was used to purify the RNA molecules and DNaseI treatment was used to destroy the DNA template. Alternatively, column purification was used, with DNaseI treatment, to prepare purified RNA molecules. RTqPCR was then performed, using a specific reverse transcription primer for the cDNA synthesis step. qPCR follows using a specific PCR reverse primer, forward primer and probe set. Both the reverse primer and probe anneal to the hairpin loop region of the cDNA and will not anneal to the original DNA template (if any residual or contaminating molecules enter the PCR reaction tube). The results are presented in FIG. 22, and demonstrate that the stem loop assay has further exemplified RTqPCR as a method for measuring RNA produced in IVT reactions.

### 3.5 RNA Detection by RNA Aptamer:Fluorophore Complexing

An opportunity to make a one-tube option for AR-IVT-RNA detection emerges from RNA molecule aptamer technologies. The RNA aptamers that have been exemplified in the following experiments are RNA Mango and RNA iSpinach.

### (1) RNA Mango

RNA Mango is a high-affinity RNA aptamer that binds the fluorophore, TO1-PEG-Biotin (TO1-PB) (TO-thiazole orange). Upon binding RNA Mango, the fluorescence of TO1-PEG-Biotin increases by up to 1000-fold. RNA Mango binds TO1 with a K_{D}=3.2±0.7 nM therefore low concentrations of the fluorophore can be used to detect RNA Mango. The on-rate of RNA binding to TO1 is within 30 minutes, with a slow off-rate of greater than 2 hours.

In aqueous solution, TO1 shows very low fluorescence because the bridge between its two heterocycles in not rigid. Upon binding to RNA Mango, the bridge becomes rigid and the molecule becomes strongly fluorescent. To further support the folding and stability of the RNA Mango molecule, it is often produced together with a scaffold structure. The sequence for the scaffold structure has been included in the DNA templates used in this study.

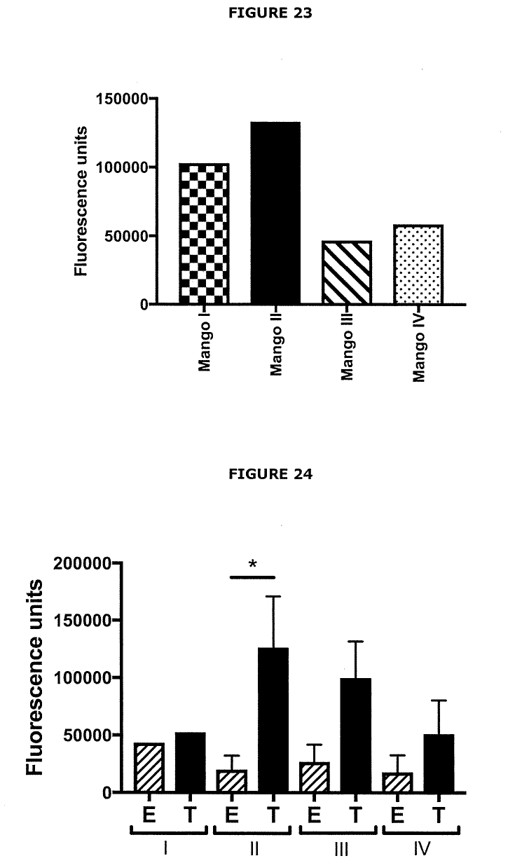

There are several forms of RNA Mango, named Mango I, II, III and IV. All four of these RNA Mango forms have been exemplified using the AR/ARE/enhancer in vitro transcription assay (sequences included in Appendix material). IVT reactions were assembled and activated with testosterone (4 nM). The IVT reactions were incubated at 30° C. for 1 h, then diluted in RNA Mango binding buffer supplemented with TO1-PB (40 nM). The binding reaction continued for 25 mins at 25° C. The results are presented in FIG. 23 and show testosterone-activated AR-ARE/enhancer IVT assay generates RNA Mango aptamer molecules that can be detected by TO1-PB fluorescence.

Next the level of RNA Mango aptamer generated from testosterone-activated reactions compared to ethanol controls was measured for each form of Mango. IVT reactions were assembled and activated with testosterone (4 nM) or ethanol (as control, 0.1% v/v final concentration). The IVT reactions were incubated at 30° C. for 1 h, then diluted in RNA Mango binding buffer supplemented with TO1-PB (40 nM). The binding reaction continued for 25 mins at 25° C. The results are presented in FIG. 24 and show the Mango variant with the highest fold difference between baseline expression (ethanol) and induced levels (testosterone) was Mango II. Accordingly, Mango II was used in subsequent experimentation.

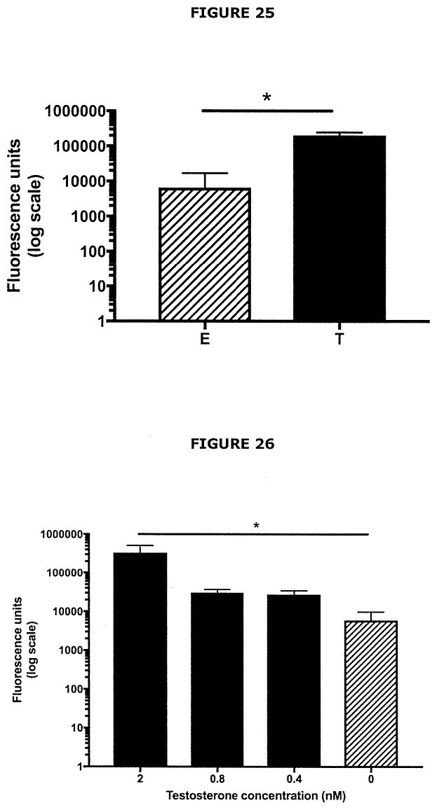

It was next necessary to determine the reproducibility of the Mango II result with testosterone versus baseline expression. The experiment was repeated three times, on consecutive days. These data are shown in FIG. 25, and demonstrate the testosterone specific activation of Mango II aptamer compared to ethanol control.

The IVT reactions so far have been activated with 4 nM testosterone which is adequate for measuring the male physiological range of 7.8-29.4 nM. However, the sensitivity range of cell-based bioassays is subnM so it was questioned whether the IVT assay could detect subnM concentrations of testosterone. IVT reactions were prepared and activated with 2, 0.8, 0.4 nM T, and compared to ethanol (0.1% v/v). The results are presented in FIG. 26 and demonstrate that an Assay Prototype 2 configured with Mango II aptamer for ligand activation could be detected in the subnM range.

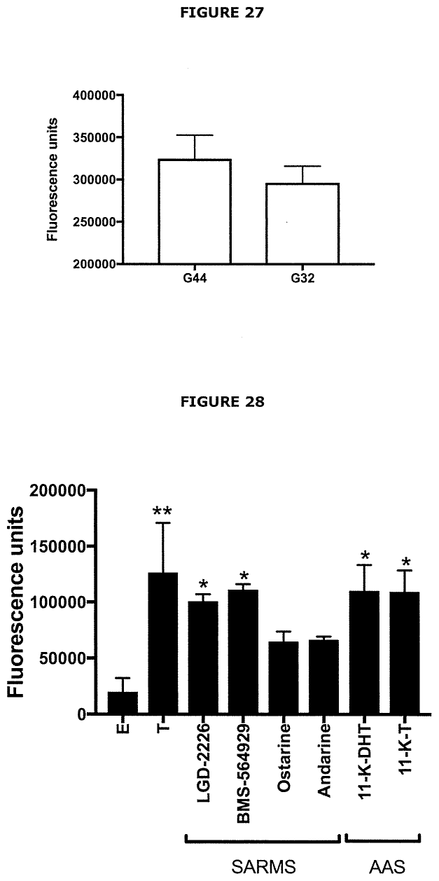

Next it was investigated whether the Assay Prototype 2 RNA Mango aptamer assay could measure androgenic activity in equine plasma samples. Two gelding samples (known as G44 and G32) have been tested in (a) a cell-based bioassay (HEK293), (b) Prototype 2 RTqPCR assay, (c) Prototype 2 direct readout assay, where G44 has consistently shown higher androgenic activity than G32. These two samples were tested in the Assay Prototype 2 RNA Mango aptamer assay. The results are presented in FIG. 27, and again show that G44 has higher androgenic activity than G32, consistent with findings from established cell-based bioassays and with other Assay Prototype 2 configurations.

Next it was investigated if the Prototype 2 Mango RNA aptamer assay was able to detect androgenic anabolic steroids (AAS) and selective androgen receptor modulators (SARMs). Four different SARMS, recently detected as substances of abuse in the equine racing industry, LGD-2226, BMS-564929, Ostarine, and Andarine, were tested in the AR-ARE/enhancer-RNA Mango aptamer assay. Two different AAS, 11-keto testosterone and 11-keto dihydrotestosterone, were also tested. IVT reactions were prepared and activated with one of the six different androgenic molecules (i.e. LGD-2226, BMS-564929, Ostarine, Andarine, 11-K-DHT and 11-K-T) or testosterone. Ethanol was used as the baseline control. These data are presented in FIG. 28, and clearly show that detection of these androgenic molecules using this particular activity/activation assay configuration. Although these six androgenic molecules represent only a small percentage of the SARMs and AAS available for use as substances for use as sports doping agents, it shows that the AR-induced IVT reaction is responsive to non-endogenous androgens.

(2) RNA iSpinach

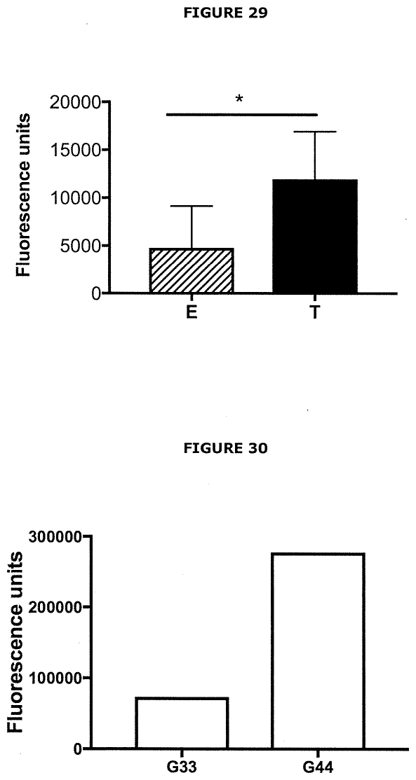

RNA iSpinach is an RNA aptamer that binds the fluorophore, 3,5-difluoro-4-hydroxybenzylidene imidazolinone, (DFHBI). Upon binding RNA iSpinach, the fluorescence of DFHBI measurably increases. RNA iSpinach binds DFHBI with an affinity of 1.18 μM. Similar to the RNA Mango experiments, the level of RNA iSpinach aptamer generated from testosterone-activated reactions compared to baseline expression (ethanol control) was measured. The only difference between the IVT reactions was the switching out of the DNA Mango template to the DNA iSpinach template. The DNA template had iSpinach expressed with the F30 scaffold. IVT reactions were assembled and activated with testosterone (4 nM) or ethanol (0.1% v/v final concentration). The IVT reactions were incubated at 30° C. for 1 h, then diluted in RNA iSpinach binding buffer supplemented with DFHBI (40 μM). The binding reaction continued for 25 mins at 37° C. The results are presented in FIG. 29, and clearly show that testosterone activated IVT reactions produced more iSpinach aptamer than the Ethanol controls.

Having showed the iSpinach reactions were responsive to T, IVT reactions were assembled and activated with equine plasma samples, G33 or G44. Both samples have previously been tested with cell-based bioassays and G33 has been shown to have lower androgenic bioactivity than G44. The IVT reactions were incubated at 30° C. for 1 h, then diluted in RNA iSpinach binding buffer supplemented with DFHBI (40 μM). The binding reaction continued for 25 mins at 37° C. Fluorescence was then read at 405 nm excitation and 498 nm emission. The results are presented in FIG. 30, and show that the IVT-iSpinach assay reactions are capable of detecting androgenic activity in equine plasma samples. Further, these data confirm previous established observation that G44 has higher androgenic activity than G33, consistent with findings from the cell-based bioassays.

### Example 4

### Assay Prototype 3: Androgen Receptor Binds Directly to DNA

Applicants further determined that the endogenous androgenic molecule, testosterone, could induce the binding of AR to the ARE/enhancer DNA template in activity assay system.

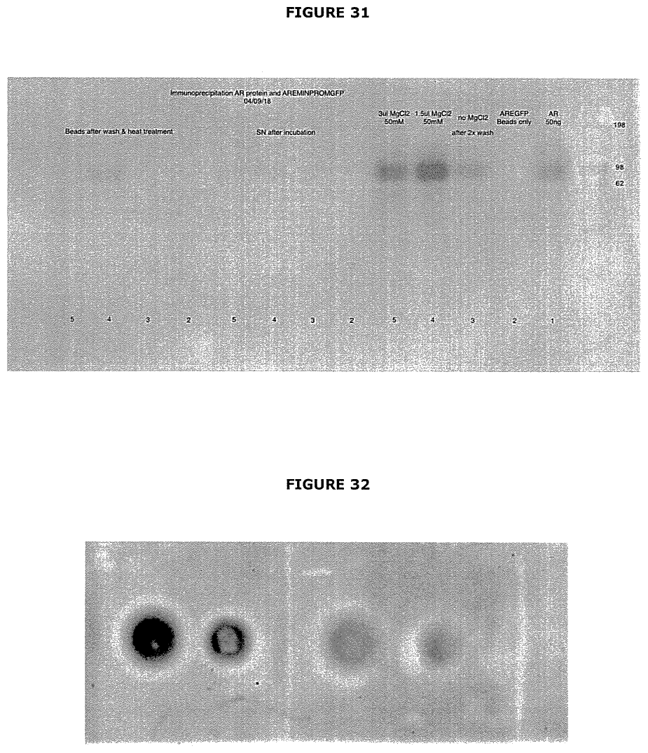

The AREminpromGFP DNA template was immobilized on magnetic beads. The beads (equivalent to 100 ng AREminpromGFP DNA) were washed in transcription buffer using a magnetic stand. Supernatant was removed and the beads were resuspended in 25 μL transcription buffer, and 100 ng recombinant AR and 5 nM testosterone was added. The reaction was incubated for 1 hour at 30° C. The transcription buffer was supplemented with no, 1.5 μL or 3 μL MgCl₂. After the incubation, supernatant was removed from the beads, and the beads were washed twice with transcription buffer, before resuspended in 20 μL transcription buffer, Western blot loading buffer and reducing agent. At this point, the samples were heated to 95° C. to break the DNA/bead bond and the supernatant removed to a fresh tube that contained 20 μl transcription buffer, Western blot loading buffer and reducing agent. The samples were then subjected to PAGE (150V, 1.5 h) and transferred to PDVF membrane (1 h 100V). Blocking was completed in PBSTM 5% for 1 h at room temperature before exposed to primary AR antibody in PBSTM overnight at 4° C. The PDVF membrane was washed 3× for 5 mins in PBST, and then incubated with anti-mouse IgG conjugated to horse radish peroxidase (HRP) in PBSTM for 4 h. The blot was again washed three times for 5 mins in PBST. HRP was visualized using WestPico for 5 mins. The results are presented in FIG. 31, and show AR binds to the AREGFP DNA template.

Next it was determined if testosterone-activated more AR to bind to the ARE-DNA template relative to ethanol (vehicle control). As a further control, the binding of AR to an ERE-DNA template was tested. The ARE DNA template was incubated in the presence of transcription buffer with recombinant AR, and as a control, an estrogen response element (ERE) DNA template was also incubated with recombinant AR. Testosterone (5 nM) or ethanol (0.1% v/v) was used to activate AR to bind to DNA template. The AR/ARE complex was then fixed onto nitrocellulose before AR was targeted using a primary antibody to AR, and then a horse radish peroxidase-conjugated secondary antibody.

The dot blot presented in FIG. 32 shows more AR binding with testosterone stimulation relative to the Ethanol control. It also shows that AR did not bind to the ERE template.

In summary, the data presented in this example show the in vitro coupled reaction of recombinant AR protein and ARE-DNA template successfully showed the binding of AR to the androgen response element. Further, the experiments have shown that testosterone induces more AR to bind to the ARE. The specificity of AR binding to its own response element is demonstrated by the poor binding of AR to the Estrogen Response Element.

### Example 5

### Supplementary Information for Assay Prototype 1

### 5.1 Generation of the DNA Template

The androgen response element/enhancer sequence used to generate Assay Prototype 1 is as follows:

SEQ ID NO: 15: Sense strand
ACTCTGGAGGAACATATTGTATCGATTAAGCTTAGAACAGTTTGTAACGA
GCTCGTTACAAACTGTTCTAGCTCGTTACAAACTGTTCTAAGCTCAAGCT
TA
SEQ ID NO: 16: Antisense strand
ATGAGACCTCCTTGTATAACATAGCTAATTCGAATCTTGTCAAACATTGC
TCGAGCAATGTTTGACAAGATCGAGCAATGTTTGACAAGATTCGAGTTCG
AA

### 5.2 In Vitro Transcription/In Vitro Translation Reaction

The following reaction mixes were prepared:

### Reaction Mix #1 [Test]

i. HeLa cell extract*

ii. recombinant AR*

iii. reaction buffer*

iv. pARE/epAGFP DNA template

v. 40 nM testosterone

### Reaction Mix #2 [Vehicle Control]

1. HeLa cell extract

2. recombinant AR

3. reaction buffer

4. pARE/epAGFP DNA template

5. 0.1% v/v ethanol

### Reaction Mix #3 [Negative Control]

1. HeLa cell extract

2. recombinant AR

3. reaction buffer

4. 0.1% v/v ethanol

### Reaction Mix #4 [Negative Control]

1. HeLa cell extract

2. reaction buffer

3. pARE/epAGFP DNA template

4. 0.1% v/v ethanol

### Reaction Mix #5 [Positive Control]

1. HeLa cell extract

2. reaction buffer

3. pCMV-GFP DNA template

4. 0.1% v/v ethanol

*HeLa cell extract, recombinant AR and reaction buffer were sourced from ThermoFisher Scientific.

As negative controls, Reaction Mix #3 was assembled without the DNA template (Reaction #3), Reaction Mix #4 was assembled without AR as HeLa cells are reported to not contain AR (this has been verified using Western analysis).

As a positive control, Reaction Mix #5 was assembled with DNA control plasmid (pCMV-GFP) from the IVT kit (ThermoFisherScientific).

The various reaction mixtures were incubated at 37° C. for 5 h. Fluorescence readings to detect amount of expressed GFP were made using a standard 96-well fluorimeter (488 nm/525 nm).

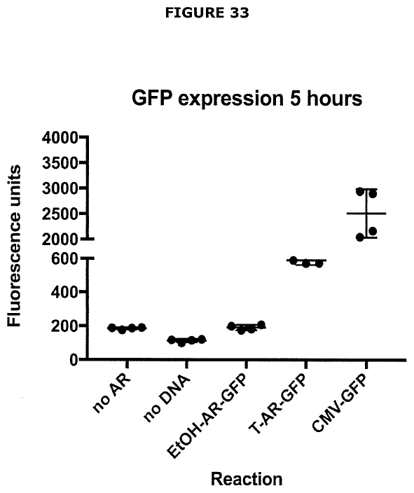

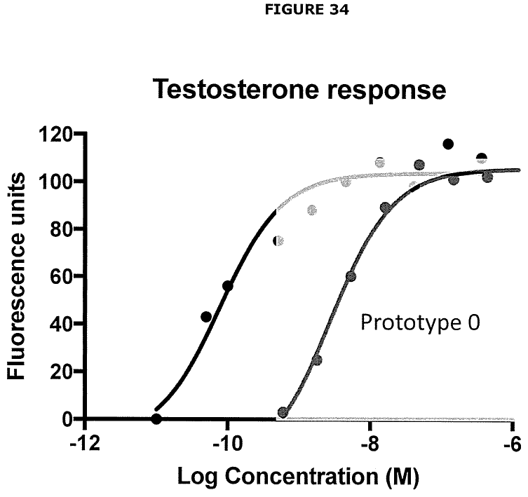

The results are shown in FIG. 33. These data show green fluorescent protein was expressed in the AR/ARE/enhancer assay when stimulated by testosterone (Reaction Mix #1), but not ethanol (Reaction Mix #2). All negative controls showed low fluorescence readings (Reaction Mix #3; Reaction Mix #4). There was measurable fluorescence in the ethanol control indicating low basal GFP expression from the minimal promoter, pA. The level was similar to that measured for the no AR control, again indicating basal RNAPII expression of GFP from pA. The no DNA template control showed absolute baseline level. The fluorescence measured in this reaction is attributable to the autofluorescence of the HeLa cell extract.

The amount of GFP produced by the inducible AR/ARE/enhancer template was far below that of the positive control, CMV-GFP (Reaction Mix #5). CMV is a strong promoter so should produce high levels of GFP. Subsequently, the Assay Prototype 1 has been further optimized to improve the dynamic range (increasing GFP output; data not shown).

5.3 Determining the Sensitivity (EC₅₀) of Assay Prototype 1(v1)

The next step was to evaluate a concentration range across which the assay could measure Testosterone. Multiple Reaction Mix #1s above were set up and used to test Testosterone from 1 μM to 1 nM. Data was used to generate a sigmoidal dose response curve. The results are shown in FIGS. 34 and 35.

FIG. 34 shows the relative sensitivity of Assay Prototype 1, as compared Assay Prototype 0 (i.e. cell-based assay).

FIG. 35 shows the EC₅₀ for Assay Prototype 2 as 7.9×10⁻¹¹M compared to the EC₅₀ for Assay Prototype 0 as 5×10⁻⁹M. This represents an almost 100-fold increase in sensitivity over the cell-based assay (Assay Prototype 0).

5.4 Determining the Specificity of Assay Prototype 1(v1)

The steroid hormone family includes estradiol (E2) and progesterone (P), and both at high doses can activate AR. Therefore, Applicants next tested if Assay Prototype 2(v1) could be activated by estradiol (E2) and progesterone (P) within a physiological range. Testing from 4 nM to 1 μM, Applicants found that both P and E2 could activate AR however only when concentrations were in the μM range. Importantly, this response was marginal and well below that measured for T at this same concentration. Note, that the basal fluorescence for this system is ˜190 units (determined in sections 5.2 and 5.3 above)

### Example 6

### Supplementary Information for Assay Prototype 2

### 6.1 In Vitro Transcription (IVT) Reaction

Assay Prototype 2 does not require the synthesis of a protein (translation) and is based only on an IVT after androgen activation of its specific receptor, the androgen receptor (AR).

The following reaction mixes were prepared:

### Reaction Mix #6

1. HeLa cell extract

2. reaction buffer

3. NTPs and MgCl₂

4. DNA template

5. Recombinant AR

6. 100 ng testosterone

### Reaction Mix #7

1. HeLa cell extract

2. reaction buffer

3. NTPs and MgCl₂

4. DNA template

5. Recombinant AR

6. 0.1% v/v ethanol

The reaction mixes were incubated at 37° C. for 1 hour during which time mRNA was synthesized from the DNA template.

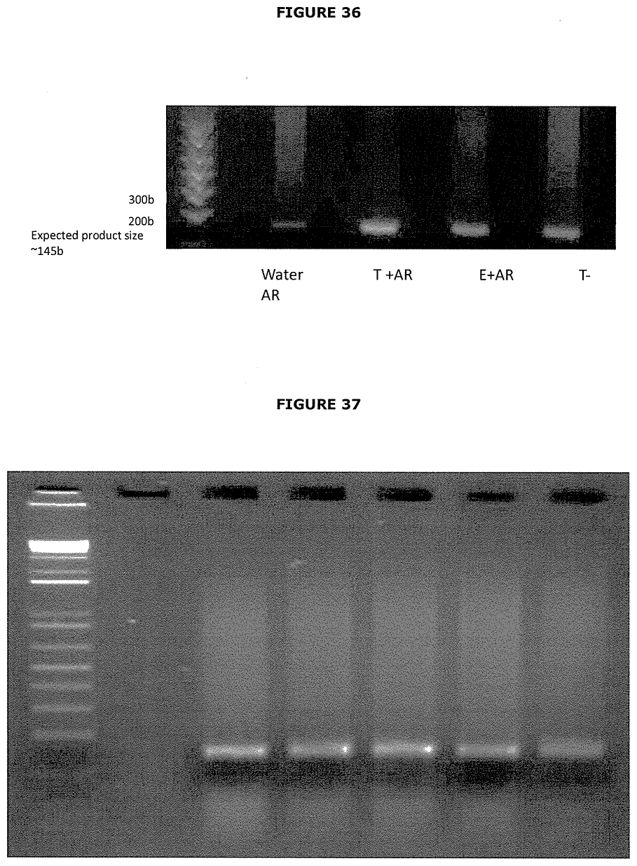

Reverse transcription-PCR (RT-PCR) was used to convert the mRNA into a DNA product. Initially, this was visualized using 2% agarose gel electrophoresis (FIG. 36).

This was the first indication that the IVT reaction would work. RT-PCR with luciferase specific primers showed a stronger band (indicative of more RNA produced) after AR was activated by T, compared to both controls. There was baseline product in both controls.

These experiments were repeated on three consecutive days, all with the same result (gels not shown). These data show that IVT is consistently activated when testosterone (T) binds androgen receptor (AR) and induces AR to initiate transcription at the MMTV.

The next step was to determine if a testosterone dose response could be seen to show that there was some dynamic range within the assay. Using 100, 50, 25 and 12.5 ng of testosterone versus ethanol, RT-PCR showed decreasing DNA output indicative of decreasing mRNA produced with less activation of androgen by lower testosterone concentrations (FIG. 37).

### 6.2. IVT Components

The IVT components used to show T+AR activation of transcription were from a commercially available kit, the HeLaScribe kit. From this kit, the AR-T IVT reaction used the HeLa cell extract and the reaction buffer.

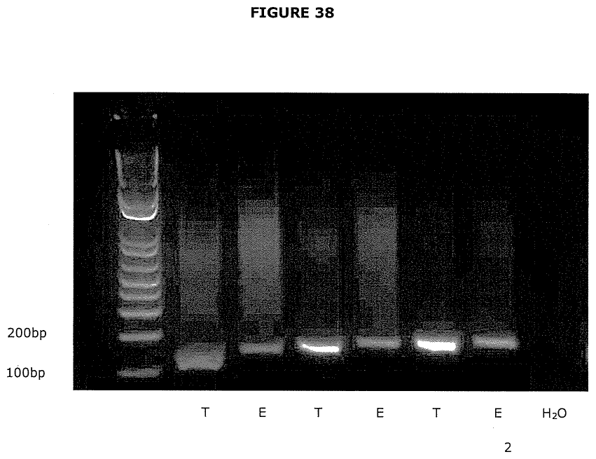

A HeLa cell extract was generated in the laboratory using cultured HeLa cells. The in house developed extract was tested in an AR-T IVT reaction (FIG. 38).

These experiments were repeated n=3 times to demonstrate reproducibility.

Ultimately, the goal is to use RTqPCR rather than RT-PCR. AR-T reaction versus AR+T IVT reactions were completed as in FIG. 38 above. The RNA was then extracted, a reverse transcription step completed to generate cDNA, and a 1:100 dilution of the cDNA was tested in a SYBRgreen-based RTqPCR assay (TaKara), using the same gene specific primers used for RTPCR.

The mean C_{T} value for AR-T reactions was 21.78. The mean C_{T} value for AR+T was 18.03. This represents an increase in expression of 13.5-fold.

### 6.3 Assay Optimization

### 6.3.1 Hormone Steroid Receptor Cofactors

In a cell, AR is held in an inactive state by protein-protein interaction with heat shock protein 90 (HSP90). When an androgen enters the cell, it binds to AR and this ligand-protein interaction causes the dislocation of HSP90 from AR. In the assay being developed, HSP90 was added to ensure that only liganded-AR activated MMTV-luciferase transcription.

IVT reactions were performed that included HSP90, with the control being no HSP90 added. RNA extraction was then completed, followed by an RT reaction. SYBRgreen-based qPCR (TaKara) showed CT value of 15.37 without HSP90, and 17.08 with HSP90.

When activated with T, no HSP90 showed 10.86, while with HSP90 14.24.

These findings show repression of the background by ˜4-fold with HSP90 and increase the activation to ˜16-fold.

The IVT reaction was incubated for 1 hour at 37° C. To determine if increasing the time of incubation to 2 hours led to increased mRNA and a larger difference between ethanol and testosterone was trialed. CT values after IVT, RNA extraction, RTqPCR shows that there was only an increase by about 4-fold after this time. The standard protocol of 1 hour for 37° C. will be adopted for assay development.

The IVT reaction can be MgCl₂ sensitive. Therefore, it was tested whether increasing MgCl₂ concentrations increased mRNA output. IVT reactions were set up with 3, 4, 5, 6 and 7 nM MgCl₂. After 1 hour at 37° C., mRNA was extracted, and RTqPCR was performed. The CT values showed that 3 nM at 25.75 was lower than for any other MgCl₂ concentration (29.38, 26.35, 27.95 and 26.22 for 4, 5, 6 and 7 nM, respectively). Therefore, the standard protocol of using 3 nM MgCl₂ in the IVT reaction was adopted for assay development.

### 6.3.2 Optimization of Cell/Cell-Free Extract

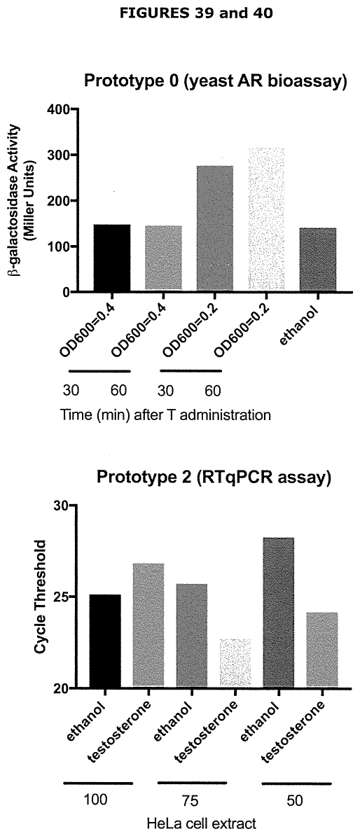

Applicant demonstrated that with cell-based bioassays, it is absolutely essential that the number of cells assayed remain below a certain threshold. For example, for Assay Prototype 0 (Yeast AR bioassay) to function optimally, the optical density (OD) of the cell culture must be less than 0.2 for the assay to differentiate AR activation from background noise. If the OD exceeds 0.2, there is no ability of the assay to show differences between test samples over that of the baseline controls. This is shown in FIG. 39. For these results, a thoroughbred race horse was administered testosterone at time 0. At times 30- and 60-minutes, blood samples were taken and analyzed for AR bioactivity by incubating the yeast AR bioassay cells in the presence of 5% race horse serum. If the yeast cells were too confluent, e.g. OD0.4, neither 30- or 60 minutes showed increased AR bioactivity above that of the negative control (ethanol) sample. However, if the cells were at the optimal confluency, OD0.2, then both the 30- and 60 minute samples showed increased AR bioactivity relative to the negative control. Also, the 60-minute sample showed more activity than the 30-minute sample. It is difficult to control cell growth, especially in the presence of serum that contains many growth factors. If the cells overgrow, the experiment has to be stopped and re-started, empirically estimating the starting cell number.

With Assay Prototype 2, it is possible to precisely optimize the cell extract component. The cell extract component can be tested for activity. Once defined as activity units, a number of units of cell component extract activity can be added to each reaction thereby defining the stoichiometry of the reaction. That cell extract concentration is an important consideration as seen in FIG. 40. For these results, Assay Prototype 2 cell-free reactions were prepared and activated with either 100 ng testosterone or an equal volume of ethanol that acted as the negative vehicle control. For each pair of cell-free reactions (testosterone and ethanol), a titrated amount of HeLa cell extract was added (100, 75 or 50 μg protein/ml). The results demonstrate that when 100 μg HeLa extract was added to the reaction, testosterone activation could not be differentiated from negative control. As the HeLa cell extract concentration decreased (75 μg and then 50 μg), there was an obvious and measurable difference between testosterone and the negative control. This is evident because there are differences detected in cycle threshold values, with the threshold value for testosterone being lower than that of ethanol indicating more starting RNA molecules. This allows for the to determination of the optimal concentration of HeLa cell extract to add to the cell-free reaction. The activity of this concentration is then determined and defined as activity units. All future reactions can then be assembled to contain the defined amount of activity units. This step removes the ambiguity of cell growth and subsequent discrepancies in repeated measures inherent with cell-based bioassays.

### 6.4 RNA Extraction

### 6.4.1 Reaction to RT-PCR

Initially, it was tested whether it was possible to simply use the IVT reaction as an mRNA sample in a RT step, followed by the PCR step.

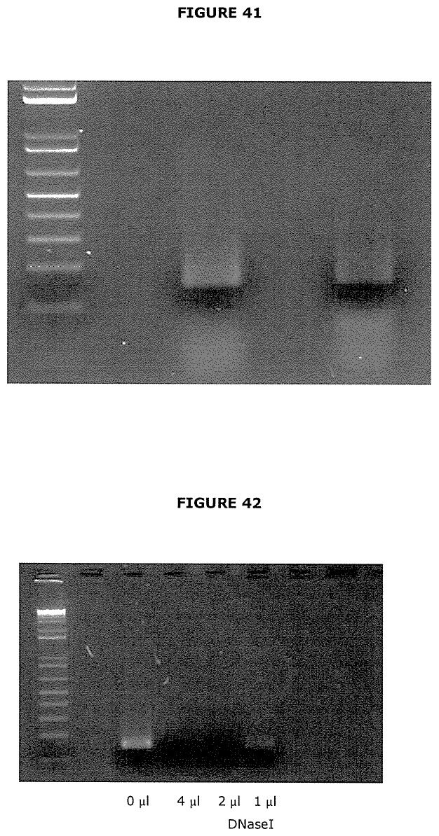

A crude RT-PCR reaction showed that a PCR band was obtained (FIG. 41).

While the PCR was not optimized, it showed that DNA was produced, and permits a no RNA extraction step.

### 6.4.2 DNase Treatments

DNaseI was used to destroy the DNA template prior to the RT-PCR step. The number of DNaseI units required for full destruction of the DNA was empirically evaluated (FIG. 42) using increasing volumes of DNaseI. From the results, 4 μL was chosen to use in the IVT reaction. However, it failed to eliminate the DNA template most likely due to high salt concentrations of the IVT reaction buffer. DNaseI was switched to Turbo DNase (ThermoScientific) because it is more tolerant of high salt conditions. Results show DNA template was destroyed and no PCR product was produced (FIG. 43).

### 6.4.3 IVT-RTqPCR Single Step

The next approach was to revisit RTqPCR directly from the IVT reaction. A crude reaction shown in FIG. 40 proved it was possible however required optimization. Using the Cells-to-C_{T} reaction enzyme/mixture (ThermoFisher Scientific), IVT reactions were performed with AR activated by T (100 ng) or ethanol (as control). DNase treatment followed then RTqPCR was performed using the one-step approach. The threshold cycle (C_{T}) values were ethanol 21.99, testosterone 19.80 (first attempt), ethanol 22.21, testosterone 19.98 (second attempt) then after adjusting the reaction volume to move the CT values >20 where they are more accurate, ethanol 34.5, testosterone 24.29. This represents a 1184-fold activation of T-AR induced mRNA synthesis.

Accordingly, proof-of-principle had been established for an androgen receptor Assay Prototype 2, whereby an IVT reaction can be activated by liganded-AR and the subsequent RNA can be detected by RTqPCR.

### Example 7

### Supplementary Information for Aptamer:Fluorophore Assay Prototype 2

### 7.1 Introduction

This study used the fluorogenic RNA aptamer iSpinach and a commercially available dye that mimics the natural fluorophore of green fluorescent protein (GFP), 3,5-difluoro-4-hydroxybenzylidene imidazolinone (DFHBI).

A DNA template was engineered that had the androgen response element (ARE) upstream of a minimal promoter element and DNA sequence/s coding for the iSpinach fluorogenic RNA aptamer. Androgen detection was demonstrated in vitro using the DNA template and the androgen receptor (AR) that together drive the transcription of the iSpinach fluorogenic RNA aptamer only in the presence of androgen.

### 7.2 Methods

An in vitro transcription reaction was performed combining 1× transcription buffer (20 mM HEPES pH 7.9, 100 mM KCl, 20% glycerol), 3 mM MgCl₂, 10 mM NTPs, 50 ng recombinant AR protein, 20 U RNase Out, 100 ng DNA template and 60 μg HeLa cell extract, (Promega) made up to 25 μL final volume with nuclease-free water. Testosterone (1 μL) or Ethanol as vehicle control (1 μL) was added to the reaction mix before it was incubated for 1 h at 30° C.

The DNA template was either (A) Enhancer/ARE TK minimal promoter 4×iSpinach (ARE4×iSpinach) with linker between each Spinach sequence or (B) enhancer/ARE TK minimal promoter with a 32mer linker sequence before a F30 Scaffold sequence, the iSpinach sequence, then the rest of the F30 Scaffold (ARE-F30iSpinach).

Following the in vitro transcription reaction, 2 μL fluorophore DFHBI (200 μM) and 73 μL fluorescence buffer (200 mM KCl, 10 mM NaHPO₄ pH7.2, 0.05% Tween-20) was added to a final volume of 100 μL. The binding reaction was incubated at 37° C. for 25 minutes in the dark.

The reaction was measured in black, clear bottom, 96-well plates on a Fluroskan (Thermofisher) at an excitation wavelength of 460 nm and detection of the emission maxima at 505 nm, with a bandwidth of 15-25 nm.

For the control reaction, DNA template was added to the transcription reaction without AR and without NTPs. This is the no-RNA generation control and tests for DFHBI binding to DNA and cell extract auto-fluorescence.

The DNA templates were synthesized (GeneART, ThermoFisher) and subcloned into plasmid vector, pMA. This plasmid has ampicillin resistance. Competent E. coli were transformed with the plasmid vector to allow plasmid amplification and purification. Plasmid DNA was linearized by restriction endonuclease digest using the enzyme PvuI and the product cleaned up by column-purification.

### 7.3 Results

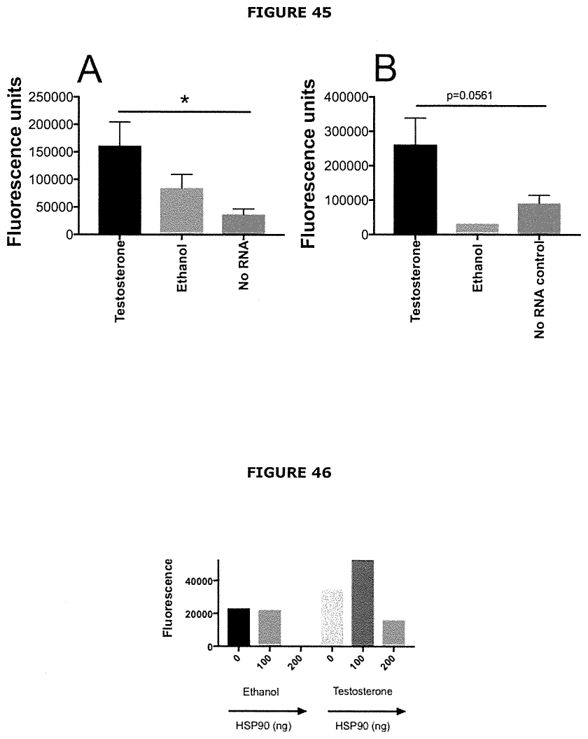

The results are presented in FIG. 45 which shows that testosterone-activated AR reactions had higher fluorescence than the ethanol and no-RNA control providing direct evidence that more fluorogenic RNA aptamer was synthesized due to presence of testosterone, relative to the ethanol or the no RNA control reactions.

The testosterone-activated AR reactions showed increased fluorescence for the two DNA templates tested: (A) ARE4×iSpinach; and (B) ARE-F30iSpinach.

### 7.4 Conclusion

The results show for the first time the synthesis of a fluorogenic RNA aptamer to detect the in vitro testosterone activation of AR and its subsequent ARE-directed RNA transcription.

Applicants have also engineered reporter constructs comprising other fluorogenic RNA aptamers (e.g.) Mango I, II, III and IV, and used its aptamer specific dye, thiazole orange (TO1), to demonstrate equivocal utility as a detection means (Example 3).

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts disclosed herein may be resorted to by those skilled in the art, and that such modifications and variations are considered to be within the scope of this invention as described herein, and as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other examples are within the following claims. In addition, where features or aspects of the invention are described in terms of Markush groups, those skilled in the art will recognize that the invention is also thereby described in terms of any individual member or subgroup of members of the Markush group.

1. A test kit for screening a test sample for the presence of a ligand, which ligand is capable of forming a complex with a steroid hormone receptor and eliciting a genomic response when in a cell, the test kit comprising:
(i) a hormone receptor that forms a receptor-ligand complex with a ligand from the test sample; and
(ii) a nucleic acid response element that is bound by the receptor-ligand complex; and
(iii) a detection means for detecting binding between the receptor-ligand complex and the nucleic acid response element; and optionally
wherein, the presence of a ligand in the test sample is determined when the sample is combined with the test kit and binding between the receptor-ligand complex and the nucleic acid response element is detected.

2. The test kit according to claim 1, wherein the relative amount of steroid hormone receptor to nucleic acid response element in the test kit is x:1, where x is the amount of steroid hormone receptor and is defined as [0.2≤x≤20].

3. The test kit according to claim 1 or claim 2, wherein the test kit further comprises a steroid hormone receptor cofactor that prevents the steroid hormone receptor from binding to the nucleic response element.

4. The test kit according to claim 3, wherein the steroid hormone receptor cofactor is selected from the group consisting of heat shock protein 70, heat shock protein 40, heat shock protein 90, p23, heat shock protein organizing protein (Hop), 48 kD Hip protein, p60, and FKBP52.

5. The test kit according to any one of claims 1 to 4, wherein the nucleic acid response element is operably linked to a reporter element, and binding of the receptor-ligand complex to the nucleic acid response element is determined by interrogating transcription or translation of the reporter element.

6. A test kit for screening a test sample for the presence of a ligand, which ligand is capable of forming a complex with a steroid hormone receptor and eliciting a genomic response when in a cell, the test kit comprising:
(i) a hormone receptor that forms a receptor-ligand complex with a ligand from the test sample; and
(ii) a nucleic acid response element that is bound by the receptor-ligand complex; and
(iii) a reporter construct operably linked to the nucleic acid response element; and optionally
(iv) a steroid hormone receptor cofactor, transcription machinery, steroid metabolism machinery and/or a cell-free extract
wherein, the reporter construct is activated when the receptor-ligand complex binds to the nucleic acid response element, and
wherein, the presence of a ligand in the sample is determined when the sample is combined with the test kit and transcription of the reporter construct is detected.

7. The test kit according to claim 6, wherein the relative amount of steroid hormone receptor to nucleic acid response element in the test kit is x:1, where x is the amount of steroid hormone receptor and is defined as [0.2≤x≤20].

8. The test kit according to claim 6 or claim 7, wherein the steroid hormone receptor cofactor is selected from the group consisting of heat shock protein 70, heat shock protein 40, heat shock protein 90, p23, heat shock protein organizing protein (Hop), 48 kD Hip protein, p60, and FKBP52.

9. The test kit according to any one of claims 6 to 8, wherein the reporter construct is comprised of a promoter sequence and a reporter construct, and the promoter sequence is activated when the nucleic acid response element is bound by the receptor-ligand complex.

10. The test kit according to any one of claims 6 to 9, wherein the reporter construct comprises a sequence encoding an RNA aptamer which is capable of binding to and enhancing fluorescence of a fluorophore.

11. The test kit according to claim 10, wherein the RNA aptamer is selected from Spinach, Spinach 2, iSpinach, Broccoli and Mango including Mango I-IV.

12. The test kit according to claim 10 or claim 11, wherein the fluorophore is selected from 3,5-difluoro-4-hydroxybenzylidene imidazolinone (DFHBI) and thiazole orange 1 (TO-1).

13. The test kit according to any one of claims 1 to 12, wherein the steroid hormone receptor is selected from the group consisting of an androgen receptor, estrogen receptor, progesterone receptor, mineralocorticoid receptor, and glucocorticoid receptor.

14. The test kit according to any one of claims 1 to 13, wherein the ligand is a performance enhancing drug and/or steroid, or wherein the ligand is of an unknown chemical structure.

15. The test kit according to any one of claims 1 to 14, wherein the sample is a biological sample derived from an animal selected from the group consisting of equine, canine, camelid, bovine, porcine, ovine, caprine, avian, simian, murine, leporine, cervine, piscine, salmonoid and human.

16. An assay method for detecting a ligand in a sample, which ligand is capable of forming a complex with a steroid hormone receptor and eliciting a genomic response when in a cell, the assay method comprising the steps of:
(i) providing assay reagents comprising:
a. a hormone receptor that forms a receptor-ligand complex with a ligand from the test sample; and
b. a nucleic acid response element that is bound by the receptor-ligand complex; and
c. a detection means for detecting binding between the receptor-ligand complex and the nucleic acid sequence; and optionally
d. a steroid hormone receptor cofactor, transcription and/or translation machinery, steroid metabolism machinery and/or a cell-free extract
(ii) combining the test sample with the assay reagents
wherein, the presence of a ligand in the sample is determined when the sample is combined with the assay reagents and binding between receptor-ligand complex and the nucleic acid response element is detected.

17. An assay method according to claim 16, wherein the sample is a biological sample derived from an animal and the presence of a ligand in the sample is indicative of doping in the animal.

18. An article of manufacture for determining sports doping in an animal, the article of manufacture comprising a test kit according to any one of claims 1 to 15 together with instructions for detecting the presence of ligand in a sample derived from the animal, wherein the presence of a ligand in the sample is indicative of doping in the animal.


SEQUENCE LISTING (19 sequences)

SEQ ID NO: 1 - DNA, 15 bp, Artificial Sequence
  Androgen Receptor Response Element Sense Strand
  misc_feature (7)..(9): n = any amino acid
ggtacannntgttct

SEQ ID NO: 2 - DNA, 15 bp, Artificial Sequence
  Androgen Receptor Response Element Antisense Strand
  misc_feature (7)..(9): n = any amino acid
ccatgtnnnacaaga

SEQ ID NO: 3 - DNA, 15 bp, Artificial Sequence
  Estrogen Receptor Alpha Response Element Sense Strand
  misc_feature (7)..(9): n = any amino acid
aggtcannntgacct

SEQ ID NO: 4 - DNA, 15 bp, Artificial Sequence
  Estrogen Alpha Response Element Antisense Strand
  misc_feature (7)..(9): n = any amino acid
tccagtnnnactgga

SEQ ID NO: 5 - DNA, 15 bp, Artificial Sequence
  Estrogen Receptor Beta Response Element Sense Strand
  misc_feature (7)..(9): n = any amino acid
aggtcannntgacct

SEQ ID NO: 6 - DNA, 15 bp, Artificial Sequence
  Estrogen Receptor Beta Response Element Antisense Strand
  misc_feature (7)..(9): n = any amino acid
tccagtnnnactgga

SEQ ID NO: 7 - DNA, 15 bp, Artificial Sequence
  Progesterone Receptor Alpha Response Element Sense Strand
  misc_feature (7)..(9): n = any amino acid
agaacannntgttct

SEQ ID NO: 8 - DNA, 15 bp, Artificial Sequence
  Progesterone Receptor Alpha Response Element Antisense Strand
  misc_feature (7)..(9): n = any amino acid
tcttgtnnnacaaga

SEQ ID NO: 9 - DNA, 15 bp, Artificial Sequence
  Progesterone Receptor Beta Response Element Sense Strand
  misc_feature (7)..(9): n = any amino acid
agaacannntgttct

SEQ ID NO: 10 - DNA, 15 bp, Artificial Sequence
  Progesterone Receptor Beta Response Element Antisense Strand
  misc_feature (7)..(9): n = any amino acid
tcttgtnnnacaaga

SEQ ID NO: 11 - DNA, 15 bp, Artificial Sequence
  Mineralcorticoid Receptor Response Element Sense Strand
  misc_feature (7)..(9): n = any amino acid
agaacannntgttct

SEQ ID NO: 12 - DNA, 15 bp, Artificial Sequence
  Mineralcorticoid Receptor Response Element Antisense Strand
  misc_feature (7)..(9): n = any amino acid
tcttgtnnnacaaga

SEQ ID NO: 13 - DNA, 15 bp, Artificial Sequence
  Glucocorticoid Receptor Response Element Sense Strand
  misc_feature (7)..(9): n = any amino acid
agaacannntgttct

SEQ ID NO: 14 - DNA, 15 bp, Artificial Sequence
  Glucocorticoid Receptor Response Element Antisense Strand
  misc_feature (7)..(9): n = any amino acid
tcttgtnnnacaaga

SEQ ID NO: 15 - DNA, 102 bp, Artificial Sequence
  Androgen Response Element/Enhancer Sense Strand
actctggaggaacatattgtatcgattaagcttagaacagtttgtaacgagctcgttacaaactgttctagctcgttacaaactgttctaagctcaagctta

SEQ ID NO: 16 - DNA, 102 bp, Artificial Sequence
  Androgen Response Element/Enhancer Antisense Strand
atgagacctccttgtataacatagctaattcgaatcttgtcaaacattgctcgagcaatgtttgacaagatcgagcaatgtttgacaagattcgagttcgaa

SEQ ID NO: 17 - DNA, 69 bp, Artificial Sequence
  iSpinach RNA Aptamer Sequence
aggagtacggtgagggtcgggtccagtaggtacgcctactgttgagtagagtgtgggctccgtactccc

SEQ ID NO: 18 - DNA, 470 bp, Artificial Sequence
  Androgen Response Elelment/Enhancer GFP Sequence (optimized for UTP and CTP)
cgcgtactctggaggaacatattgtatcgattaagcttagaacagtttgtaacgagctcgttacaaactgttctagctcgttacaaactgttctaagctcaagcttagggcccaattcgaagtacttcgcatattaaggtgacgcgtgtggcctcgaacaccgagcgaccctgcagcgacccgcttaaaagatccagccaccagatctttgtcgatcctaccatccactcgacacacccgccagcggccgctgccaagcttccgagctctcgaattcaaaggaggtacccaccatgactgctctgtctgtgcgtgcgatactgttcgagaaggagatcccgtacatctctgtgttggaaggagatgtcgaagggatcaaattcatcatcaacgcagaacgaacgggtgacgcaacgaccgcaacgatcatcgccatatacatttgtaccacgggcgatttgcctgtctaa

SEQ ID NO: 19 - DNA, 380 bp, Artificial Sequence
  Androgen Response Element/Enhancer + iSpinach RNA Aptamer Sequence
tggagaacagcctgttctccatctagatggagaacagcctgttctccatctagatggagaacagcctgttctccatctagagccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagaagcttcgaatcgcgaattcgcccaccatggggagacagcctacgagcctgagcctccagtcttgccatgtgtatgtgggtacgaaggagaggagaggaagaggagagtacccacatactctgatgatccttcgggatcattcatggcaa